(19) United States
(12) Patent Application Publication
(10) Pub. No.: US 2010/0222311 A1
(43) Pub. Date: Sep. 2, 2010

(54) SOLID FORMULATIONS OF CRYSTALLINE COMPOUNDS

(75) Inventors: Markus Thommes, Duesseldorf (DE); Rodolfo Pinal, West Lafayette, IN (US); Teresa M. Carvajal, West Lafayette, IN (US)

Correspondence Address: BARNES & THORNBURG LLP, 11 SOUTH MERIDIAN, INDIANAPOLIS, IN, 46204, US

(73) Assignee: PURDUE RESEARCH FOUNDATION, West Lafayette, IN (US)

(21) Appl. No.: 12/682,938

(22) PCT Filed: Oct. 17, 2008
(86) PCT No.: PCT/US2008/080327

§ 371 Date: Apr. 14, 2010

Related U.S. Application Data
(60) Provisional application No. 60/981,185, filed on Oct. 19, 2007.
(60) Provisional application No. 61/038,943, filed on Mar. 24, 2008.

(51) Int. Cl. A61K 31/585; A61K 31/192; A61K 31/337; A61K 31/343; A61K 31/497; A61K 31/4166; A61P 29/00; A61P 25/00; A61P 19/00; A61P 11/00; A61P 3/00; A61P 35/00

(52) U.S. Cl. 514/173; 514/570; 514/449; 514/462; 514/254.07; 514/391

(57) ABSTRACT

Described herein are formulations of active pharmaceutical ingredients, where the active pharmaceutical ingredients or drugs are included in a solid suspension with one or more solid additives. The formulations described herein are useful for formulating any drug or active pharmaceutical ingredient, including those that have limited solubility in organic and/or aqueous solvent systems.

### CROSS REFERENCE TO RELATED APPLICATIONS

This application claims the benefit under 35 U.S.C. §119(e) of U.S. Provisional Application Ser. No. 60/981,185, filed Oct. 19, 2007, and U.S. Provisional Application Ser. No. 60/038,943, filed Mar. 24, 2008, the disclosures of which are hereby incorporated herein by reference.

### TECHNICAL FIELD

The present invention relates to the field of formulations.

### BACKGROUND AND SUMMARY OF THE INVENTION

The improvement of the bioavailability of drugs, and especially poorly soluble drugs has been the focus of a significant body of pharmaceutical research. Many different approaches across the pharmaceutical industry have been reported for addressing this issue. In the particular arena of solid formulations for tablet, capsules, dispersible powders, and the like, a typical approach is to increase the bioavailability of the drug using surfactants and other hydratropic substances. Recently, solid dispersions have been reported where drugs are dispersed in a solid carrier matrix. In those dispersions, the drug may be amorphous for rapid dissolution, or in some cases it may retain some degree of crystallinity. However, it is well established that the carrier matrix is advantageously 100% amorphous in those dispersion. Those solid dispersions are prepared by dissolving the drug in a highly water soluble polymer matrix, and at the end of the manufacturing process, the polymer matrix, and often both the drug and the polymer matrix, are in an amorphous solid state, which accelerates the dissolution rate from the dosage form. Moreover, it is conventionally accepted that when such solid dispersions are prepared, the detection of the presence of high crystallinity in the drug, or any crystallinity of the carrier matrix, results in the discard of that formulated batch. Accordingly, it has been accepted that crystallinity in the carrier matrix is a deleterious property that negatively affects the dissolution rate and ultimate release of the drug from a solid dispersion. With those constraints, such solid dispersion formulations also have the drawbacks of limitations on the drug load and the instability of amorphous materials preventing storage of the formulated material over time, or under typical environmental conditions of heat and humidity.

It has been discovered that formulations of active pharmaceutical ingredients, including those active pharmaceutical ingredients that have limited solubility in either or both of pharmaceutically acceptable organic solvent systems and pharmaceutically acceptable aqueous solvents systems, that comprise a mixture of small crystals may lead to more rapid dispersion, dissolution, and/or release of such active pharmaceutical ingredients. In general, the formulations may be characterized by the intimate mixture of small crystals of one or more active pharmaceutical ingredients and one or more water soluble solid additive. Such solid formulations are also referred to herein as solid suspensions, indicating that at least one of the active pharmaceutical ingredients and at least one of the solid additives are in a crystalline form. The crystals of both the active pharmaceutical ingredients and the solid additives are generally in the micrometer range, consistent with flowable powders. However, it is appreciated that a wide range of crystal sizes may be accommodated by the processes described herein, such as including crystals from the millimeter range to the nanometer range, and still lead to rapidly dissolving, rapidly dispersion, rapidly disintegrating, and/or rapidly releasing formulations. It is also understood that the formulations described herein may exhibit improved storage capability, in terms of length of storage time, and/or storage conditions, such as relative humidity and temperature.

In one illustrative embodiment pharmaceutical compositions comprising a solid suspension of about 5-95% by weight of one or more active pharmaceutical ingredients and about 95-5% by weight of one or more pharmaceutically acceptable water soluble additives are described. In one aspect, at least one of the solid additives has a melting temperature less than the melting temperature of the active pharmaceutical agent. In another aspect, at least a portion of at least one of the active pharmaceutical ingredients is present as crystals in the solid suspension. In another aspect, at least a portion of at least one of the solid additives is present as crystals in the solid suspension.

In another illustrative embodiment, pharmaceutical compositions are described wherein the additives are selected from pharmaceutically acceptable polyhydroxy compounds, hydroxy carboxylic acids, and/or polyhydroxy carboxylic acids.

In another illustrative embodiment, pharmaceutical compositions are described wherein the additives are selected from pharmaceutically acceptable reduced carbohydrates, sugar alcohols, and hydroxy carboxylic acids.

### BRIEF DESCRIPTION OF THE DRAWINGS

FIG. 1. Process parameters of extrusion used in preparing formulation Gri10: (a) Torque [Ncm], (b) temperature [° C.] and (c) screw speed [rpm].

FIG. 2a. Dissolution profile: (a) Gri10, (b) Phe10, (c) Spi10, (d) griseofulvin, (e) phenytoin (f) spironolactone ( x̅±CI, α=0.05, n=6).

FIG. 2b. Dissolution profile: (a) Gri50, (b) Gri50 28d, (c) Gri50 90d, (d) griseofulvin, ( x̅±CI, α=0.05, n=6).

FIG. 2c. Dissolution profile extrudates with 10% griseofulvin: (a) lactic acid (b) mannitol, (c) xylitol, (d) griseofulvin powder.

FIG. 3a. Thermogram: (a) Gri10, (b) α-mannitol and (c) griseofulvin.

FIG. 3b. Thermogram: (a) Phe10, (b) α-mannitol and (c) phenytoin.

FIG. 3c. Thermogram: (a) Spi10, (b) α-mannitol and (c) spironolactone.

FIG. 3d. Thermogram: (a) Gri50, (b) α-mannitol and (c) griseofulvin.

FIG. 4a. X-Ray pattern: (a) Cyri10, (b) α-mannitol and (c) griseofulvin.

FIG. 4b. X-Ray pattern: (a) Phe10, (b) α-mannitol and (c) Phenytoin.

FIG. 4c. X-Ray pattern: (a) Spi10, (b) α-mannitol and (c) spironolactone.

FIG. 4d. X-Ray pattern: (a) Gri50, (b) α-mannitol and (c) griseofulvin.

FIG. 5a. X-Ray diffraction pattern from (a) glucose extrudate and (b) glucose.

FIG. 5b. X-Ray diffraction pattern from (a) fructose extrudate and (b) fructose.

FIG. 6a. X-Ray diffraction pattern from (a) sorbitol extrudate and (b) sorbitol.

FIG. 6b. X-Ray diffraction pattern from (a) mannitol extrudate and (b) mannitol.

FIG. 7a. X-Ray diffraction pattern from (a) xylitol extrudate and (b) xylitol.

FIG. 7b. X-Ray diffraction pattern from (a) arabitol extrudate and (b) arabitol.

FIG. 8. X-Ray diffraction pattern from (a) lactic acid extrudate and (b) lactic acid.

FIG. 9a. X-Ray diffraction pattern from (a) extrudate, (b) xylitol and (c) griseofulvin.

FIG. 9b. X-Ray diffraction pattern from (a) extrudate, (b) lactic acid and (c) griseofulvin.

FIG. 9c. DSC thermogram from (a) extrudate and (b) xylitol.

FIG. 9d. DSC thermogram from (a) extrudate and (b) lactic acid.

FIG. 10. Dissolution profiles in water at 37° C. (n=6) (a) Gri50, low shear force; (b) Gri50, high shear force; (c) Gri10, low shear force; (d) Gri10m high shear force.

### DETAILED DESCRIPTION

In one illustrative embodiment pharmaceutical compositions comprising a solid suspension of about 5-95% by weight of one or more active pharmaceutical ingredients and about 95-5% by weight of one or more pharmaceutically acceptable water soluble additives are described. In one aspect, at least one of the solid additives has a melting temperature less than the melting temperature of the active pharmaceutical agent. In another aspect, at least a portion of at least one of the active pharmaceutical ingredients is present as crystals in the solid suspension. In another aspect, at least a portion of at least one of the solid additives is present as crystals in the solid suspension.

In another illustrative embodiment, pharmaceutical compositions are described wherein the additives are selected from pharmaceutically acceptable polyhydroxy compounds, hydroxy carboxylic acids, and/or polyhydroxy carboxylic acids.

In another illustrative embodiment, pharmaceutical compositions are described wherein the additives are selected from pharmaceutically acceptable reduced carbohydrates, sugar alcohols, and hydroxy carboxylic acids.

In another embodiment, pharmaceutical compositions comprising an active pharmaceutical ingredient are described, such as those of any of the preceding embodiments, wherein the solid additive is an monomer. In another embodiment, pharmaceutical compositions comprising an active pharmaceutical ingredient are described, such as those of any of the preceding embodiments, wherein the solid additive is an oligomer. In one aspect the oligomer is a 10-mer or less. In one variation, the oligomer is a 5-mer or less. In another variation, the oligomer is a 3-mer or less. In another variation, the oligomer is a 2-mer or less. It is appreciated that each monomer of the foregoing oligomers may be the same or different. Illustrative monomers include, but are not limited to the polyhydroxy compounds, hydroxy carboxylic acids, polyhydroxy carboxylic acids, reduced carbohydrates, sugar alcohols, and hydroxy carboxylic acids described herein. In another aspect, each monomer has a molecular weight of about 1000 or less. In one variation, the molecular weight of each monomer is about 500 or less. In another variation, the molecular weight of each monomer is about 250 or less. In another variation, the molecular weight of each monomer is about 200 or less.

In particular, the solid additives described herein may be illustratively selected from, but are not limited to, arabitol, erythritol, xylitol, sorbitol, mannitol, lactic acid, malic acid, tartaric acid, citric acid, adonitol, and/or lactitol, and combinations thereof. In one variation, the solid additives described herein may be selected from mannitol, lactic acid, adonitol, xylitol, and/or sorbitol, and combinations thereof. In another variation, the solid additives described herein may be selected from xylitol, mannitol, and/or lactic acid, and combinations thereof.

In another embodiment, pharmaceutical compositions are described, such as those of any of the preceding embodiments, wherein the unformulated active pharmaceutical ingredient has a melting point of at least about 100° C. In one variation, the unformulated active pharmaceutical ingredient has a melting point of at least about 125° C. In another variation, the unformulated active pharmaceutical ingredient has a melting point of at least about 150° C. In another variation, the unformulated active pharmaceutical ingredient has a melting point of at least about 200° C. In another variation, the unformulated active pharmaceutical ingredient has a melting point of at least about 250° C.

In another embodiment, pharmaceutical compositions are described, such as those of any of the preceding embodiments, wherein the active pharmaceutical ingredient may be illustratively selected from, but are not limited to, the following, and combinations thereof:

| | CAS Reg. | | | mp |
|---|---|---|---|---|
| API Name | No. | Brand Name | Illustrative Indications | (° C.) |
| Nicotine | 54-11-5 | Nicoderm Habitrol | smoking cessation | −79 |
| Nitroglycerin | 55-63-0 | Nitro-Bid Nitrostat | angina | 13.5 |
| Chlorpromazine | 50-53-3 | Thorazine | child behavior problems psychotic | <25 |
| | | | disorders | |
| Cyclophosphamide | 50-18-0 | Cytoxan | cancer | 51.5 |
| Gemfibrozil | 25812-30-0 | Lopid | high cholesterol | 62 |
| Isosorbide dinitrate | 87-33-2 | Isordil Sorbitrate | angina | 70 |
| Ibuprofen | 15687-27-1 | Motrin Advil | arthritis menstrual cramps pain | 76 |
| Mupirocin | 12650-69-0 | Bactroban | impetigo | 77-78 |
| Anastrozole | 120511-73-1 | Arimidex | cancer | 81-82 |
| Methocarbamol | 532-03-6 | Robaxin | muscular strain | 92-94 |
| Nabumetone | 42924-53-8 | Relafen | arthritis | 80.0 |
| Carisoprodol | 78-44-4 | Soma | muscular strain | 92 |
| Ketoprofen | 22071-15-4 | Orudis Actron | arthritis menstrual cramps pain | 94 |
| | | Oruvail | | |

In another embodiment, pharmaceutical compositions are described, such as those of any of the preceding embodiments, wherein the active pharmaceutical ingredient may be illustratively selected from, but are not limited to, the following, and combinations thereof:

| | CAS Reg. | | | mp |
|---|---|---|---|---|
| API Name | No. | Brand Name | Illustrative Indications | (° C.) |
| Metaproterenol sulfate | 5874-97-5 | Alupent Metaprel | asthma | 100.0 |
| Benzoyl peroxide | 94-36-0 | Desquam-E | acne | 105 |
| | | Benzac | | |
| Meprobamate | 57-53-4 | Miltown Equanil | anxiety disorders | 105 |
| Pentoxifylline | 5/6/6493 | Trental | impaired circulation | 105.0 |
| Captopril | 62571-86-2 | Capoten | congestive heart failure high blood | 106 |
| | | | pressure | |
| Azelaic acid | 123-99-9 | Azelex | acne | 106.5 |
| Ramipril | 87333-19-5 | Altace | congestive heart failure high blood | 109 |
| | | | pressure | |
| Cisapride | 81098-60-4 | Propulsid | heartburn | 109.8 |
| Lindane | 58-89-9 | Kwell | lice | 112.5 |
| Spironolactone | 52-01-7 | Aldactone | high blood pressure | 115.0 |
| Betaxolol | 63659-19-8 | Betoptic | glaucoma | 116.0 |
| hydrochloride | | | | |

In another embodiment, pharmaceutical compositions are described, such as those of any of the preceding embodiments, wherein the active pharmaceutical ingredient may be illustratively selected from, but are not limited to, the following, and combinations thereof:

| | CAS Reg. | | | mp |
|---|---|---|---|---|
| API Name | No. | Brand Name | Illustrative Indications | (° C.) |
| Trandolapril | 87679-37-6 | Mavik | heart attack high blood pressure | 125.0 |
| Terconazole | 67915-31-5 | Terazol | candidiasis | 126.3 |
| Chlorpropamide | 94-20-2 | Diabinese | diabetes | 128 |
| Tolbutamide | 64-77-7 | Orinase | diabetes | 128.5 |
| Oxybutynin | 1508-65-2 | Ditropan | urinary tract pain | 129.5 |
| hydrochloride | | | | |
| Diazepam | 439-14-5 | Valium | alcohol withdrawal anxiety disorders | 132 |
| | | | epilepsy muscular strain | |
| Aspirin | 50-78-2 | Ecotrin Bayer | arthritis fever reduction of heart attack | 135 |
| | | Empirin | pain reduction of stroke | |
| Echothiophate iodide | 513-10-0 | Phospholine iodide | glaucoma | 138 |
| Cimetidine | 51481-61-9 | Tagamet | heartburn peptic ulcers | 142 |
| Trimipramine maleate | 521-78-8 | Surmontil | depression | 142.0 |
| Benztropine mesylate | 132-17-2 | Cogentin | Parkinson's disease | 143 |
| Ciclopirox olamine | 41621-49-2 | Loprox | fungal infections | 144.0 |
| Felodipine | 72509-76-3 | Plendil | high blood pressure | 145.0 |
| Ketoconazole | 65277-42-1 | Nizoral | fungal infections | 146 |
| Etodolac | 41340-25-4 | Lodine | arthritis pain | 146.5 |
| Salsalate | 552-94-3 | Disalcid | arthritis | 147 |
| Clotrimazole | 23593-75-1 | Gyne-Lotrimin | fungal infections | 148 |
| | | Mycelex | | |
| Nilutamide | 63612-50-0 | Nilandron | cancer | 149.0 |
| Astemizole | 68844-77-9 | Hismanal | symptomatic relief of allergies hay fever | 149.1 |

In another embodiment, pharmaceutical compositions are described, such as those of any of the preceding embodiments, wherein the active pharmaceutical ingredient may be illustratively selected from, but are not limited to, the following, and combinations thereof:

| | CAS | | | mp |
|---|---|---|---|---|
| API Name | Reg. No. | Brand Name | Illustrative Indications | (° C.) |
| Felbamate | 25451- | Felbatol | epilepsy | 151.5 |
| | 15-4 | | | |
| Haloperidol | 52-86-8 | Haldol | child behavior problems psychotic disorders tics | 151.5 |
| Omeprazole | 73590- | Prilosec | peptic ulcers | 156 |
| | 58-6 | | | |
| Indomethacin | 53-86-1 | Indocin | arthritis pain | 158 |
| Metronidazole | 443-48-1 | Flagyl | dysentery bone and joint infections CNS | 160.5 |
| | | Protostat | infections gynecologic infections lower | |
| | | | respiratory tract infections skin infections urinary | |
| | | | tract infections sexually transmitted diseases | |
| Indapamide | 26807- | Lozol | fluid retention high blood pressure | 161 |
| | 65-8 | | | |
| Warfarin sodium | 129-06-6 | Coumadin | blood clotting | 161.0 |
| Econazole nitrate | 68797- | Spectazole | fungal infections | 162.0 |
| | 31-9 | cream | | |
| Dipyridamole | 58-32-2 | Persantine | blood clotting | 163 |
| Famotidine | 76824- | Pepcid | heartburn peptic ulcers | 163.5 |
| | 35-6 | | | |
| Dicyclomine | 67-92-5 | Bentyl | spastic colon | 165 |
| hydrochloride | | | | |
| Itraconazole | 84625- | Sporanox | fungal infections | 166.2 |
| | 61-6 | | | |
| Leflunomide | 75706- | Arava | arthritis | 166.5 |
| | 12-6 | | | |
| Lorazepam | 846-49-1 | Ativan | anxiety disorders | 167 |
| Glyburide | 10238- | Micronase | diabetes | 169 |
| | 21-8 | DiaBeta | | |
| | | Glynase | | |
| Lactulose | 4618-18-2 | Chronulac | constipation | 169 |
| | | syrup | | |
| | | Duphalac | | |
| Acetaminophen | 103-90-2 | Tylenol | fever menstrual cramps pain | 170 |
| | | Panadol | | |
| Repaglinide | 135062- | Prandin | diabetes | 170.0 |
| | 02-1 | | | |
| Risperidone | 106266- | Risperdal | psychotic disorders | 170.0 |
| | 06-2 | | | |
| Lovastatin | 75330- | Mevacor | high cholesterol | 174.5 |
| | 75-5 | | | |
| Docusate sodium | 577-11-7 | Colace Sof- | constipation | 176 |
| | | Lax | | |
| Estradiol | 50-28-2 | Estraderm | cancer menopause osteoporosis female sex | 178.5 |
| | | Alora Climara | hormone deficiency | |
| Sulindac | 38194- | Clinoril | arthritis pain | 183 |
| | 50-2 | | | |
| Clopidogrel | 113665- | Plavix | impaired circulation reduction of heart attack | 184.0 |
| bisulfate | 84-2 | | reduction of stroke | |
| Meperidine | 50-13-5 | Demerol | pain | 187.5 |
| hydrochloride | | | | |
| Carbamazepine | 298-46-4 | Tegretol | epilepsy trigeminal neuralgia | 190.2 |
| | | Atretol Epitol | | |
| Chlorzoxazone | 95-25-0 | Parafon Forte | muscular strain | 191.5 |
| | | DSC | | |
| Hydroxyzine | 2192-20-3 | Atarax Vistaril | symptomatic relief of allergies anxiety disorders | 193.0 |
| hydrochloride | | | sedation | |
| Sulfisoxazole | 80-74-0 | Gantrisin | urinary tract infections | 193.5 |
| acetyl | | | | |
| Olanzapine | 132539- | Zyprexa | psychotic disorders | 195.0 |
| | 06-1 | | | |
| Phentermine | 1197-21-3 | Fastin Adipex- | obesity | 198.0 |
| hydrochloride | | P Lonamin | | |

In another embodiment, pharmaceutical compositions are described, such as those of any of the preceding embodiments, wherein the active pharmaceutical ingredient may be illustratively selected from, but are not limited to, the following, and combinations thereof:

| | CAS | | | mp |
|---|---|---|---|---|
| API Name | Reg. No. | Brand Name | Illustrative Indications | (° C.) |
| Ursodiol | 128-13-2 | ACTIGALL | gallstones | 203 |
| Glimepiride | 93479- | AMARYL | diabetes | 207.0 |
| | 97-1 | | | |
| Methazolamide | 554-57-4 | NEPTAZANE | glaucoma | 213.5 |
| Desoximetasone | 382-67-2 | TOPICORT | skin inflammation swelling redness | 217 |
| Hydrocortisone | 50-23-7 | CETACORT | skin inflammation swelling redness | 220 |
| | | DERMACORT | | |
| | | HYTONE | | |
| Griseofulvin | 126-07-8 | GRIS-PEG | fungal infections | 220.0 |
| | | GRISACTIN | | |
| | | FULVICIN | | |
| Trazodone | 25332- | DESYREL | depression | 223.0 |
| hydrochloride | 39-2 | | | |
| Cetirizine | 83881- | ZYRTEC | symptomatic relief of allergies hay | 225.0 |
| hydrochloride | 52-1 | | fever | |
| Prochlorperazine | 58-38-8 | COMPAZINE | anxiety disorders psychotic disorders | 228 |
| | | | vomiting and nausea | |
| Estazolam | 29975- | PROSOM | insomnia | 228.5 |
| | 16-4 | | | |
| Ipratropium bromide | 22254- | ATROVENT | asthma coughs and colds hay fever | 231 |
| | 24-6 | | | |
| Metformin | 1115-70-4 | GLUCOPHAGE | diabetes | 232.0 |
| hydrochloride | | | | |
| Methylprednisolone | 83-43-2 | MEDROL | adrenal hormone deficiency severe | 232.5 |
| | | | allergies arthritis asthma colitis | |
| | | | collagen diseases inflammatory | |
| | | | diseases lupus | |
| Levothyroxine | 51-48-9 | SYNTHROID | thyroid hormone deficiency | 235.5 |
| | | LEVOTHROID | | |
| Chlordiazepoxide | 58-25-3 | LIBRIUM | alcohol withdrawal anxiety disorders | 236.2 |
| Clonazepam | 1622-61-3 | KLONOPIN | epilepsy panic disorders | 237.5 |
| Chlorthalidone | 77-36-1 | HYGROTON | fluid retention high blood pressure | 239 |
| | | THALITONE | | |
| Hydroxychloroquine | 747-36-4 | PLAQUENIL | arthritis lupus malaria | ~240 |
| sulfate | | | | |

In another embodiment, pharmaceutical compositions are described, such as those of any of the preceding embodiments, wherein the active pharmaceutical ingredient may be illustratively selected from, but are not limited to, the following, and combinations thereof:

| | CAS | | | mp |
|---|---|---|---|---|
| API Name | Reg. No. | Brand Name | Illustrative Indications | (° C.) |
| Morphine sulfate | 64-31-3 | MS CONTIN KADIAN | pain | 250 |
| Acyclovir | 59277- | ZOVIRAX | chicken pox Herpes simplex | 255 |
| | 89-3 | | sexually transmitted diseases | |
| | | | shingles | |
| Metolazone | 17560- | ZAROXOLYN | high blood pressure | 256 |
| | 51-9 | MYKROX | | |
| Sulfacetamide sodium | 127-56-0 | SODIUM SULAMYD | eye infections | 257.0 |
| | | BLEPH-10 | | |
| Raloxifene | 84449- | EVISTA | osteoporosis | 258.0 |
| hydrochloride | 90-1 | | | |
| Trihexyphenidyl | 52-49-3 | ARTANE | Parkinson's disease | 258.5 |
| hydrochloride | | | | |
| Acetazolamide | 59-66-5 | DIAMOX | epilepsy fluid retention glaucoma | 260.5 |
| | | | congestive heart failure mountain | |
| | | | sickness | |
| Nitrofurantoin | 67-20-9 | MACRODANTIN | urinary tract infections | 263 |
| | | MACROBID | | |
| Theophylline | 58-55-9 | THEO-DUR SLO-BID | asthma | 273 |
| | | T-PHYL | | |
| Desonide | 638-94-8 | TRIDESILON | skin inflammation swelling | 274 |
| | | DESOWEN | redness | |
| Hydrochlorothiazide | 58-93-5 | HYDRODIURIL | fluid retention congestive heart | 274 |
| | | ESIDRIX | failure high blood pressure | |
| Primidone | 125-33-7 | MYSOLINE | epilepsy | 281.5 |
| Fluorouracil | 51-21-8 | EFUDEX | cancer | 283 |
| Mesalamine | 89-57-6 | ROWASA PENTASA | colitis | 283 |
| | | ASACOL | | |
| Triamcinolone acetonide | 76-25-5 | AZMACORT | asthma hay fever nasal polyps | 293 |
| | | NASACORT | | |
| Furosemide | 54-31-9 | LASIX | fluid retention congestive heart | 295 |
| | | | failure high blood pressure | |
| Fluorometholone | 426-13-1 | FML | inflammatory eye diseases | 297 |
| Dextroamphetamine | 51-63-8 | DEXEDRINE | attention deficit narcolepsy | >300 |
| sulfate | | | | |
| Clonidine hydrochloride | 4205-91-8 | CATAPRES | high blood pressure | 305.0 |
| Fluocinonide | 356-12-7 | LIDEX | skin inflammation swelling | 309 |
| | | | redness | |
| Allopurinol | 315-30-0 | ZYLOPRIM | gout kidney stones | 350 |

In another embodiment, pharmaceutical compositions are described, such as those of any of the preceding embodiments, wherein the active pharmaceutical ingredient may be illustratively selected from, but are not limited to, the following, and combinations thereof:

| | CAS Reg. | | | mp |
|---|---|---|---|---|
| API Name | No. | Brand Name | Illustrative Indications | (° C.) |
| Famciclovir | 104227- | FAMVIR | Herpes simplex shingles | 102-104 |
| | 87-4 | | | |
| Flurbiprofen | 5104-49-4 | ANSAID | arthritis pain | 110-111 |
| Flutamide | 13311- | EULEXIN | cancer | 111.5-112.5 |
| | 84-7 | | | |
| Calcitriol | 32222- | ROCALTROL | abnormal calcium levels | 111-115 |
| | 06-3 | | | |
| Zidovudine | 30516- | RETROVIR | HIV infections | 113-115 |
| | 87-1 | | | |
| Azithromycin | 83905- | ZITHROMAX | ear infections lower respiratory tract | 113-115 |
| | 01-5 | | infections skin infections upper | |
| | | | respiratory tract infections sexually | |
| | | | transmitted diseases | |
| Carvedilol | 72956- | COREG | congestive heart failure high blood | 114-115 |
| | 09-3 | | pressure | |
| Mirtazapine | 61337- | REMERON | depression | 114-116 |
| | 67-5 | | | |
| Alprostadil | 745-65-3 | CAVERJECT | impotence | 115-116 |
| | | EDEX MUSE | | |
| Clomiphene citrate | 50-41-9 | CLOMID | female infertility | 116.5-118 |
| Valsartan | 137862- | DIOVAN | high blood pressure | 116-117 |
| | 53-4 | | | |
| Beclomethasone | | | | 117-120 (dec) |
| dipropionate | | | | |
| Temazepam | 846-50-4 | RESTORIL | insomnia | 119-121 |
| Fluvoxamine | 6387-89-9 | LUVOX | obsessive-compulsive disorder | 120-121.5 |
| maleate | | | | |
| Quinapril | 82586- | ACCUPRIL | congestive heart failure high blood | 120-130 |
| hydrochloride | 55-8 | | pressure | |
| Nadolol | 42200- | CORGARD | angina high blood pressure | 124-136 |
| | 33-9 | | | |

In another embodiment, pharmaceutical compositions are described, such as those of any of the preceding embodiments, wherein the active pharmaceutical ingredient may be illustratively selected from, but are not limited to, the following, and combinations thereof:

| | CAS Reg. | | | mp |
|---|---|---|---|---|
| API Name | No. | Brand Name | Illustrative Indications | (° C.) |
| Paroxetine | 78246-49-8 | PAXIL | depression obsessive-compulsive disorder panic | 129-131 |
| hydrochloride | | | disorders | |
| Nizatidine | 76963-41-2 | AXID | peptic ulcers | 130-132 |
| Loratadine | 79794-75-5 | CLARITIN | symptomatic relief of allergies hay fever skin | 134-136 |
| | | | inflammation swelling redness | |
| Simvastatin | 79902-63-9 | ZOCOR | high cholesterol reduction of heart attack | 135-138 |
| | | | reduction of stroke | |
| Erythromycin | 114-07-8 | ERYTHROCIN | acne ear infections heart infections lower | 135-140, |
| | | ERYCETTE | respiratory tract infections skin infections upper | resolidifies |
| | | | respiratory tract infections urinary tract | with second |
| | | | infections Legionnaires' disease rheumatic fever | mp 190-193 |
| | | | sexually transmitted diseases whooping cough | |
| Quazepam | 36735-22-5 | DORAL | insomnia | 137.5-139 |
| Oxiconazole | 64211-46-7 | OXISTAT | fungal infections | 137-138 |
| nitrate | | | | |
| Salmeterol | 94749-08-3 | SEREVENT | asthma | 137-138 |
| xinafoate | | | | |
| Fluconazole | 86386-73-4 | DIFLUCAN | fungal infections | 138-140 |
| Zafirlukast | 107753- | ACCOLATE | asthma | 138-140 |
| | 78-6 | | | |
| Zolmitriptan | 139264- | ZOMIG | migraine headache | 139-141 |
| | 17-8 | | | |
| Tamoxifen | 54965-24-1 | NOLVADEX | cancer | 140-142 |
| citrate | | | | |
| Acebutolol | 34381-68-5 | SECTRAL | abnormal heart rhythms high blood pressure | mp 141-143 |
| hydrochloride | | | | |
| Selegiline | 14611-52-0 | ELDEPRYL | Parkinson's disease | 141-142 |
| hydrochloride | | | | |
| Moexipril | 82586-52-5 | UNIVASC | high blood pressure | 141-161 |
| hydrochloride | | | | |
| Enalapril | 76095-16-4 | VASOTEC | congestive heart failure high blood pressure | 143-144.5 |
| maleate | | | | |
| Flecainide | 54143-56-5 | TAMBOCOR | abnormal heart rhythms | 145-147 |
| acetate | | | | |
| Atenolol | 29122-68-7 | TENORMIN | angina heart attack high blood pressure | 146-148 |
| Tolcapone | 134308- | TASMAR | Parkinson's disease | 146-148 |
| | 13-7 | | | |
| Thiothixene | 5591-45-7 | NAVANE | psychotic disorders | 147.5-149 |
| Cyclosporine | 59865-13-3 | SANDIMMUNE | arthritis organ rejection | 148-151 |
| | | NEORAL | | |

In another embodiment, pharmaceutical compositions are described, such as those of any of the preceding embodiments, wherein the active pharmaceutical ingredient may be illustratively selected from, but are not limited to, the following, and combinations thereof:

| | CAS Reg. | | | mp |
|---|---|---|---|---|
| API Name | No. | Brand Name | Illustrative Indications | (° C.) |
| Indinavir sulfate | 157810- | CRIXIVAN | HIV infections | 150-153 (dec) |
| | 81-6 | | | |
| Nisoldipine | 63675-72-9 | SULAR | high blood pressure | 151-152 |
| Zileuton | 111406- | ZYFLO | asthma | 157-158 |
| | 87-2 | | | |
| Albuterol free base | 18559-94-9 | | asthma | 157-158 |
| Celecoxib | 184007- | CELEBREX | arthritis | 157-159 |
| | 95-2 | | | |
| Fluoxetine hydrochloride | 59333-67-4 | PROZAC | bulimia depression obsessive- | 158.4-158.9 |
| | | | compulsive disorder | |
| Dipivefrin hydrochloride | 64019-93-8 | PROPINE | glaucoma | 158-159 |
| Thioridazine | 130-61-0 | MELLARIL | psychotic disorders | 158-160 |
| hydrochloride | | | | |
| Oxaprozin | 21256-18-8 | DAYPRO | arthritis | 160.5-161.5 |
| Lamivudine | 134678- | EPIVIR | HIV infections | 160-162 |
| | 17-4 | | | |
| Didanosine | 69655-05-6 | VIDEX | HIV infections | 160-163 |
| Butoconazole nitrate | 64872-77-1 | FEMSTAT | candidiasis fungal infection | 162-163 |
| Gabapentin | 60142-96-3 | NEURONTIN | epilepsy | 162-166 |
| Propranolol hydrochloride | 318-98-9 | INDERAL | adrenal gland tumors angina | 163-164 |
| | | | migraine headache heart | |
| | | | attack abnormal heart | |
| | | | rhythms high blood pressure | |
| | | | hereditary tremors | |
| Stavudine | 3056-17-5 | ZERIT | HIV infections | 165-166 |
| Sumatriptan succinate | 103628- | IMITREX | cluster headache migraine | 165-166 |
| | 48-4 | | headache | |
| Diphenhydramine | 147-24-0 | BENADRYL | symptomatic relief of | 166-170 |
| hydrochloride | | | allergies coughs and colds | |
| | | | hay fever motion sickness | |
| | | | Parkinson's disease skin | |
| | | | inflammation swelling and | |
| | | | redness | |
| Pindolol | 13523-86-9 | VISKEN | high blood pressure | 167-171 |
| Diethylpropion | 134-80-5 | TENUATE | obesity | dec 168 |
| hydrochloride | | | | |
| Isradipine | 75695-93-1 | DYNACIRC | high blood pressure | 168-170 |
| Tetracycline | 60-54-8 | ACHROMYCIN V | acne eye infections lower | 172.5 dec |
| | | SUMYCIN | respiratory tract infections | |
| | | | upper respiratory tract | |
| | | | infections urinary tract | |
| | | | infections sexually | |
| | | | transmitted diseases | |
| Quetiapine fumarate | 111974- | SEROQUEL | psychotic disorders | 172-173 |
| | 72-2 | | | |
| Nifedipine | 21829-25-4 | PROCARDIA | angina high blood pressure | 172-174 |
| | | ADALAT | | |
| Imipramine hydrochloride | 113-52-0 | TOFRANIL | bed wetting depression | 174-175 |
| Isotretinoin | 4759-48-2 | ACCUTANE | acne | 174-175 |
| Phenobarbital | 50-06-6 | PHENOBARBITAL | epilepsy sedation | 174-178 |
| Clemastine fumarate | 14976-57-9 | TAVIST | symptomatic relief of | 177-178 |
| | | | allergies hay fever | |
| Rizatriptan benzoate | 145202- | MAXALT | migraine headache | 178-180 |
| | 66-0 | | | |
| Lansoprazole | 103577- | PREVACID | heartburn peptic ulcers | 178-182 (dec). |
| | 45-3 | | | |
| Nicardipine hydrochloride | 54527-84-3 | CARDENE | angina high blood pressure | 179-181 |
| Irbesartan | 138402- | AVAPRO | high blood pressure | 180-181 |
| | 11-6 | | | |
| Tramadol hydrochloride | 22204-88-2 | ULTRAM | pain | 180-181 |
| Nefazodone hydrochloride | 82752-99-6 | SERZONE | depression | 181.0-182.0 |
| Metoclopramide | 54143-57-6 | REGLAN | heartburn vomiting and | 182.5-184 |
| hydrochloride | | | nausea | |
| Clozapine | 5786-21-0 | CLOZARIL | psychotic disorders | 183-184 |
| Miconazole nitrate | 22832-87-7 | MONISTAT | candidiasis fungal infections | 184-185 |
| Troglitazone | 97322-87-7 | REZULIN | diabetes | 184-186 |
| Dirithromycin | 62013-04-1 | DYNABAC | lower respiratory tract | 186-189 (dec) |
| | | | infections skin infections | |
| | | | upper respiratory tract | |
| | | | infections | |
| Trimethobenzamide | 554-92-7 | TIGAN | vomiting and nausea | 187.5-190 |
| hydrochloride | | | | |
| Labetalol hydrochloride | 32780-64-6 | NORMODYNE | high blood pressure | 187-189 |
| | | TRANDATE | | |
| Doxepin hydrochloride | 1229-29-4 | SINEQUAN | depression | 188-189 |
| Benazepril hydrochloride | 86541-74-4 | LOTENSIN | high blood pressure | 188-190 |
| Flurazepam hydrochloride | 1172-18-5 | DALMANE | insomnia | 190-220 |
| Clomipramine | 17321-77-6 | ANAFRANIL | obsessive-compulsive | 191.5-192 |
| hydrochloride | | | disorder | |
| Guanabenz acetate | 23256-50-0 | WYTENSIN | high blood pressure | 192.5 (dec) |
| Bromocriptine mesylate | 22260-51-1 | PARLODEL | Parkinson's disease | 192-196 (dec) |
| Sibutramine hydrochloride | 125494- | MERIDIA | obesity | 193-195.5 |
| | 59-9 | | | |
| Fluvastatin sodium | 93957-55-2 | LESCOL | high cholesterol reduction of | 194-197. |
| | | | heart attack | |
| Clobetasol propionate | 25122-46-7 | TEMOVATE | skin inflammation swelling | 195.5-197 |
| | | CORMAX | redness | |
| Amitriptyline | 549-18-8 | ELAVIL | depression | 196-197 |
| hydrochloride | | | | |
| Cefadroxil monohydrate | 66592-87-8 | DURICEF | skin infections upper | 197 (dec). |
| | | | respiratory tract infections | |
| | | | urinary tract infections | |
| Piroxicam | 36322-90-4 | FELDENE | arthritis pain | 198-200 |

In another embodiment, pharmaceutical compositions are described, such as those of any of the preceding embodiments, wherein the active pharmaceutical ingredient may be illustratively selected from, but are not limited to, the following, and combinations thereof:

| | CAS Reg. | | | mp |
|---|---|---|---|---|
| API Name | No. | Brand Name | Illustrative Indications | (° C.) |
| Doxycycline hyclate | 24390- | DORYX | acne cholera infectious diarrhea | Chars without |
| | 14-5 | VIBRAMYCIN | dysentery eye infections lower | melting at |
| | | | respiratory tract infections rickettsiae | about 201 |
| | | | infections skin infections upper | |
| | | | respiratory tract infections urinary tract | |
| | | | infections sexually transmitted diseases | |
| Buspirone | 33386- | BUSPAR | anxiety disorder | 201.5-202.5 |
| hydrochloride | 08-2 | | | |
| Timolol | 26839- | TIMOPTIC | glaucoma | 201.5-203 |
| | 75-8 | BETIMOL | | |
| Mexiletine | ″5370- | MEXITIL | abnormal heart rhythms | 203-205 |
| hydrochloride | 01-4 | | | |
| Pilocarpine | 54-71-7 | PILOCAR | glaucoma | 204-205 |
| hydrochloride | | ISOPTO | | |
| | | CARPINE | | |
| Oxazepam | 604-75-1 | SERAX | anxiety disorders | 205-206 |
| Loracarbef | 76470- | LORABID | ear infections sinus infections skin | 205-215 (dec) |
| | 66-1 | | infections upper respiratory tract | |
| | | | infections urinary tract infections | |
| Diltiazem | 33286- | CARDIZEM | angina high blood pressure | 207.5-212 |
| hydrochloride | 22-5 | DILACOR | | |
| | | TIAZAC | | |
| Medroxyprogesterone | 71-58-9 | PROVERA | uterine bleeding regulation of menstrual | 207-209 |
| acetate | | CYCRIN | cycle | |
| Ampicillin | 69-53-4 | OMNIPEN | ear infections lower respiratory tract | 208 dec |
| | | PRINCIPEN | infections upper respiratory tract | |
| | | TOTACILLIN | infections urinary tract infections | |
| | | | sexually transmitted diseases | |
| Glipizide | 29094- | GLUCOTROL | diabetes | 208-209 |
| | 61-9 | | | |
| Levobunolol | 27912- | BETAGAN | glaucoma | 209-211 |
| hydrochloride | 14-7 | | | |
| Diflunisal | 22494- | DOLOBID | arthritis pain | 210-221 |
| | 42-4 | | | |
| Donepezil | 120011- | ARICEPT | Alzheimer's disease | 211-212 (dec) |
| hydrochloride | 70-3 | | | |
| Alclometasone | 66734- | ACLOVATE | skin inflammation swelling redness | 212-216 |
| dipropionate | 13-2 | | | |
| Nortriptyline | 894-71-3 | PAMELOR | depression | 213-215 |
| hydrochloride | | AVENTYL | | |
| Guanfacine | 29110- | TENEX | high blood pressure | 213-216 |
| hydrochloride | 48-3 | | | |
| Procanbid | 51-06-9 | PROCAN SR | abnormal heart rhythms | 214-216 |
| | | PROCANBID | | |
| Desipramine | 58-28-6 | NORPRAMIN | depression | 215-216 |
| hydrochloride | | | | |
| Venlafaxine | 99300- | EFFEXOR | depression | 215-217 |
| hydrochloride | 78-4 | | | |
| Cyclobenzaprine | 6202- | FLEXERIL | muscular strain | 216-218 |
| hydrochloride | 23-9 | | | |
| Lamotrigine | 84057- | LAMICTAL | epilepsy | 216-218 |
| | 84-1 | | | |
| Zalcitabine | 7481- | HIVID | HIV infections | 217-218 |
| | 89-2 | | | |
| Mometasone furoate | 83919- | ELOCON | skin inflammation swelling redness | 218-220 |
| | 23-7 | | | |
| Cefprozil | 92665- | CEFZIL | sinus infections skin infections upper | 218-220 (dec) |
| | 29-7 | | respiratory tract infections | |
| Gentamicin sulfate | 1405- | GARAMYCIN | eye infections | 218-237 |
| | 41-0 | OPHTHALMIC | | |
| Clarithromycin | 81103- | BIAXIN | lower respiratory tract infections sinus | 220 dec |
| | 11-9 | | infections skin infections upper | |
| | | | respiratory tract infections peptic ulcers | |
| Sulfasalazine | 599-79-1 | AZULFIDINE | arthritis colitis | 220 dec |
| Enoxacin | 74011- | PENETREX | urinary tract infections sexually | 220-224 |
| | 58-8 | | transmitted diseases | |
| Diflorasone diacetate | 33564- | PSORCON | skin inflammation swelling redness | 221-223 (dec) |
| | 31-7 | | | |
| Loperamide | 34552- | IMODIUM | diarrhea | 222-223 |
| hydrochloride | 83-5 | | | |
| Levofloxacin | 100986- | LEVAQUIN | lower respiratory tract infections sinus | 225-227 (dec) |
| | 85-4 | | infections skin infections urinary tract | |
| | | | infections | |
| Azelastine | 79307- | ASTELIN | hay fever | 225-229 |
| hydrochloride | 93-0 | | | |
| Budesonide | 51333- | RHINOCORT | symptomatic relief of allergies hay | 226 dec |
| | 22-3 | | fever skin inflammation swelling | |
| | | | redness | |
| Alprazolam | 28981- | XANAX | anxiety disorders panic disorders | 228-228.5 |
| | 97-7 | | | |
| Tamsulosin | 106463- | FLOMAX | benign prostate enlargement | 228-230 |
| hydrochloride | 17-6 | | | |
| Bumetanide | 28395- | BUMEX | fluid retention congestive heart failure | 230-231 |
| | 03-1 | | | |
| Mefenamic acid | 61-68-7 | PONSTEL | menstrual cramps | 230-231 |
| Promethazine | 58-33-3 | PHENERGAN | symptomatic relief of allergies hay | 230-232 (some |
| hydrochloride | | | fever motion sickness sedation vomiting | dec) |
| | | | and nausea | |
| Dihydroergotamine | 6190- | MIGRANAL | migraine headache | 230-235 |
| mesylate | 39-2 | | | |
| Ondansetron | 103639- | ZOFRAN | vomiting and nausea | 231-232 |
| | 04-9 | | | |
| Betamethasone | 5593- | DIPROLENE | skin inflammation swelling redness | 232 dec |
| dipropionate | 20-4 | | | |
| Flavoxate | 3717- | URISPAS | urinary tract pain | 232-234 |
| hydrochloride | 88-2 | | | |
| Prednisone | 53-03-2 | DELTASONE | adrenal hormone deficiency severe | dec 233-235 |
| | | ORASONE | allergies arthritis asthma colitis collagen | |
| | | | diseases inflammatory diseases lupus | |
| Bupropion | 31677- | WELLBUTRIN | depression smoking cessation | 233-234 |
| hydrochloride | 93-7 | ZYBAN | | |
| Triazolam | 28911- | HALCION | insomnia | 233-235 |
| | 01-5 | | | |
| Naratriptan | 143388- | AMERGE | migraine headache | 237-239 |
| hydrochloride | 64-1 | | | |
| Olsalazine sodium | 15722- | DIPENTUM | colitis | 240 (dec) |
| | 48-2 | | | |
| Cromolyn sodium | 16110- | CROLOM | hay fever inflammatory eye diseases | 241 dec |
| | 51-3 | | | |
| Ropinirole | 91374- | REQUIP | Parkinson's disease | 241-243 |
| hydrochloride | 20-8 | | | |
| Trifluoperazine | 440-17-5 | STELAZINE | anxiety disorders psychotic disorders | 242-243 |
| hydrochloride | | | | |
| Sertraline | 79617- | ZOLOFT | depression obsessive-compulsive | 243-245 |
| hydrochloride | 96-2 | | disorder panic disorders | |
| Naproxen sodium | 26159- | ANAPROX | arthritis fever gout inflammatory | 244-246 |
| | 34-2 | ALEVE | diseases menstrual cramps pain | |
| | | NAPRELAN | | |
| Tocainide | 35891- | TONOCARD | abnormal heart rhythms | 246-247 |
| hydrochloride | 93-1 | | | |
| Terbutaline sulfate | 23031- | BRETHINE | asthma | 246-248 |
| | 32-5 | BRICANYL | | |
| | | BRETHAIRE | | |
| Nevirapine | 129618- | VIRAMUNE | HIV infections | 247-249 |
| | 40-2 | | | |
| Digoxin | 20830- | LANOXIN | congestive heart failure abnormal heart | 249 dec |
| | 75-5 | | rhythms | |

In another embodiment, pharmaceutical compositions are described, such as those of any of the preceding embodiments, wherein the active pharmaceutical ingredient may be illustratively selected from, but are not limited to, the following, and combinations thereof:

| | CAS | | | mp |
|---|---|---|---|---|
| API Name | Reg. No. | Brand Name | Illustrative Indications | (° C.) |
| Finasteride | 98319- | PROPECIA | baldness benign prostate enlargement | 252-254 |
| | 26-7 | PROSCAR | | |
| Ofloxacin | 82419- | FLOXIN | gynecologic infections lower respiratory | 254 dec |
| | 36-1 | | tract infections skin infections urinary tract | |
| | | | infections sexually transmitted diseases | |
| Estropipate | 7280-37-7 | OGEN ORTHO- | osteoporosis female sex hormone deficiency | 254.5-256 |
| | | EST | | |
| Pemoline | 2152-34-3 | CYLERT | attention deficit | 256 dec |
| Alendronate | 129318- | FOSAMAX | osteoporosis Paget's disease | 257-262.5 |
| sodium | 43-0 | | | |
| Dexamethasone | 50-02-2 | DECADRON | adrenal hormone deficiency severe allergies | 262-264 |
| | | TABLETS | arthritis asthma colitis collagen diseases hay | |
| | | | fever inflammatory diseases lupus | |
| Fluticasone | 90566- | FLONASE | symptomatic relief of allergies asthma hay | 272-273 (dec) |
| | 53-3 | FLOVENT | fever | |
| Naltrexone | 16676- | REVIA | alcohol withdrawal narcotic withdrawal | 274-276 |
| hydrochloride | 29-2 | | | |
| Penciclovir | 39809- | DENAVIR | Herpes simplex | 275-277 |
| | 25-1 | | | |
| Terazosin | 70024- | HYTRIN | high blood pressure benign prostate | 278-279 |
| hydrochloride | 40-7 | | enlargement | |
| Tacrine | 1684-40-8 | COGNEX | Alzheimer's disease | 283-284 |
| hydrochloride | | | | |
| Diclofenac | 15307- | VOLTAREN | arthritis menstrual cramps pain | 283-285 |
| sodium | 79-6 | CATAFLAM | | |
| Yohimbine | 65-19-0 | YOCON | impotence | 289 dec |
| hydrochloride | | YOHIMEX | | |
| Lomefloxacin | 98079- | MAXAQUIN | lower respiratory tract infections urinary | 290-300 (dec) |
| hydrochloride | 52-8 | | tract infections | |
| Betaine | 107-43-7 | CYSTADANE | high homocysteine levels | 293 dec |
| anhydrous | | | | |
| Pramipexole | 104632- | MIRAPEX | Parkinson's disease | 296-301 |
| hydrochloride | 25-9 | | | |
| Methyldopa | 555-30-6 | ALDOMET | high blood pressure | 300 dec |
| Ciprofloxacin | 93107- | CIPRO | infectious diarrhea bone and joint infections | 318-320 |
| hydrochloride | 08-5 | | lower respiratory tract infections sinus | |
| | | | infections skin infections upper respiratory | |
| | | | tract infections urinary tract infections | |
| Adapalene | 106685- | DIFFERIN | acne | 319-322 |
| | 40-9 | | | |
| Chlorothiazide | 58-94-6 | DIURIL | fluid retention high blood pressure | 350 dec |

In another embodiment, pharmaceutical compositions are described, such as those of any of the preceding embodiments, wherein the active pharmaceutical ingredient may be illustratively selected from, but are not limited to, the following, and combinations thereof:

| | CAS | | |
|---|---|---|---|
| API Name | Reg. No. | Brand Name | Illustrative Indications |
| Acarbose | 56180- | PRECOSE | diabetes |
| | 94-0 | | |
| Amcinonide | 51022- | CYCLOCORT | skin inflammation swelling redness |
| | 69-6 | | |
| Amlodipine besylate | 88150- | NORVASC | angina high blood pressure |
| | 42-9 | | |
| Amoxicillin | 26787- | AMOXIL TRIMOX | ear infections lower respiratory tract infections skin |
| | 78-0 | WYMOX | infections upper respiratory tract infections sexually |
| | | | transmitted diseases peptic ulcers |
| Atorvastatin calcium | 134523- | LIPITOR | high cholesterol |
| | 03-8 | | |
| Benzonatate | 104-31-4 | TESSALON | coughs and colds |
| Cefaclor | 53994- | CECLOR | ear infections lower respiratory tract infections skin |
| | 73-3 | | infections upper respiratory tract infections urinary |
| | | | tract infections |
| Cefixime | 79350- | SUPRAX | ear infections lower respiratory tract infections upper |
| | 37-1 | | respiratory tract infections |
| Ceftibuten | 97519- | CEDAX | ear infections upper respiratory tract infections |
| | 39-6 | | |
| Cefuroxime axetil | 64544- | CEFTIN | ear infections lower respiratory tract infections |
| | 07-6 | | rickettsiae infections skin infections upper respiratory |
| | | | tract infections urinary tract infections sexually |
| | | | transmitted diseases |
| Cephalexin | 105879- | KEFLEX KEFTAB | bone and joint infections lower respiratory tract |
| hydrochloride | 42-3 | | infections skin infections urinary tract infections |
| Cerivastatin sodium | 143201- | BAYCOL | high cholesterol |
| | 11-0 | | |
| Choline magnesium | 64425- | TRILISATE | arthritis pain |
| trisalicylate | 90-7 | | |
| Citalopram | 59729- | CELEXA | depression |
| hydrobromide | 32-7 | | |
| Clorazepate | 57109- | TRANXENE | anxiety disorders |
| dipotassium | 90-7 | | |
| Chlorhexidine | 18472- | PERIDEX | gingivitis |
| gluconate | 51-0 | | |
| Clindamycin | 24729- | CLEOCIN T | acne |
| phosphate | 96-2 | | |
| Cyproheptadine | 969-33-5 | PERIACTIN | severe allergies symptomatic relief of allergies coughs |
| hydrochloride | | | and colds |
| Disopyramide | 22059- | NORPACE | abnormal heart rhythms |
| phosphate | 60-5 | | |
| Doxazosin mesylate | 77883- | CARDURA | high blood pressure benign prostate enlargement |
| | 43-3 | | |
| Fexofenadine | 138452- | ALLEGRA | symptomatic relief of allergies hay fever |
| hydrochloride | 21-8 | | |
| Flunisolide | ″3385- | AEROBID | asthma |
| | 03-03 | NASALIDE | |
| Fosfomycin | 78964- | MONUROL | urinary tract infections |
| tromethamine | 85-9 | | |
| Fosinopril sodium | 88889- | MONOPRIL | high blood pressure |
| | 14-9 | | |
| Hydromorphone | 71-68-1 | DILAUDID | pain |
| hydrochloride | | | |
| Hyoscyamine sulfate | 620-61-1 | LEVSIN | spastic colon |
| | | ANASPAZ | |
| | | LEVBID | |
| Isosorbide | 16051- | IMDUR ISMO | angina |
| mononitrate | 77-7 | MONOKET | |
| Ketorolac | 74103- | TORADOL | pain |
| tromethamine | 07-4 | | |
| Latanoprost | 130209- | XALATAN | glaucoma |
| | 82-4 | | |
| Lisinopril | 76547- | ZESTRIL | heart attack high blood pressure |
| | 98-3 | PRINIVIL | |
| Losartan potassium | 124750- | COZAAR | high blood pressure |
| | 99-8 | | |
| Meclizine | 36236- | ANTIVERT | motion sickness |
| hydrochloride | 67-6 | BONINE | |
| Methylergonovine | 57432- | METHERGINE | postpartum bleeding |
| maleate | 61-8 | | |
| Methylphenidate | 298-59-9 | RITALIN | attention deficit narcolepsy |
| hydrochloride | | | |
| Metoprolol tartrate | 56392- | LOPRESSOR | angina heart attack high blood pressure |
| | 17-7 | TOPROL-XL | |
| Methotrexate | 59-05-2 | RHEUMATREX | arthritis cancer psoriasis |
| Minocycline | 13614- | MINOCIN | acne cholera dysentery lower respiratory tract |
| hydrochloride | 98-7 | DYNACIN | infections rickettsiae infections skin infections upper |
| | | | respiratory tract infections urinary tract infections |
| | | | sexually transmitted diseases |
| Misoprostol | 59122- | CYTOTEC | peptic ulcers |
| | 46-2 | | |
| Montelukast sodium | 151767- | SINGULAIR | asthma |
| | 02-1 | | |
| Nedocromil sodium | 69049- | TILADE | asthma |
| | 74-7 | | |
| Nelfinavir mesylate | 159989- | VIRACEPT | HIV infections |
| | 65-8 | | |
| Penicillin V | 132-98-9 | BEEPEN-VK PEN- | dental infections ear infections heart infections lower |
| potassium | | VEE | respiratory tract infections skin infections upper |
| | | | respiratory tract infections rheumatic fever |
| Phenelzine sulfate | 156-51-4 | NARDIL | depression |
| Phenazopyridine | 136-40-3 | PYRIDIUM | urinary tract pain |
| hydrochloride | | | |
| Phenytoin sodium | 630-93-3 | DILANTIN | epilepsy |
| Pravastatin sodium | 81131- | PRAVACHOL | high cholesterol reduction of heart attack |
| | 70-6 | | |
| Prazosin | 19237- | MINIPRESS | high blood pressure |
| hydrochloride | 84-4 | | |
| Prednisolone sodium | 125-02-0 | PEDIAPRED | adrenal hormone deficiency severe allergies arthritis |
| phosphate | | | asthma colitis collagen diseases inflammatory diseases |
| | | | lupus |
| Propafenone | 54063- | RYTHMOL | abnormal heart rhythms |
| | 53-5 | | |
| Quinidine | 27555- | CARDIOQUIN | abnormal heart rhythms |
| polygalacturonate | 34-6 | | |
| Ranitidine bismuth | 128345- | TRITEC | peptic ulcers |
| citrate | 62-0 | | |
| Ritonavir | 155213- | NORVIR | HIV infections |
| | 67-5 | | |
| Saquinavir | 127779- | FORTOVASE | HIV infections |
| | 20-8 | | |
| Sildenafil citrate | 171599- | VIAGRA | impotence |
| | 83-0 | | |
| Sucralfate | 54182- | CARAFATE | peptic ulcers |
| | 58-0 | | |
| Tazarotene | 118292- | TAZORAC | acne psoriasis |
| | 40-3 | | |
| Tobramycin | 32986- | TOBREX AKTOB | eye infections |
| | 56-4 | | |
| Tolmetin sodium | 64490- | TOLECTIN | arthritis pain |
| | 92-2 | | |
| Valacyclovir | 124832- | VALTREX | shingles |
| hydrochloride | 27-5 | | |
| Valproic acid | 99-66-1 | DEPAKENE | epilepsy |
| | | DEPAKOTE | |
| Verapamil | 152-11-4 | CALAN ISOPTIN | angina abnormal heart rhythms high blood pressure |
| hydrochloride | | VERELAN | |

In another embodiment, pharmaceutical compositions are described, such as those of any of the preceding embodiments, wherein the active pharmaceutical ingredient may be illustratively selected from, but are not limited to, ibuprofen, paclitaxol, griseofulvin, itraconazole, phenytoin, spironolactone, and combinations thereof.

In another embodiment, pharmaceutical compositions are described, such as those of any of the preceding embodiments, wherein the active pharmaceutical ingredient is in at least a partially crystalline form, where the presence and degree of crystallinity may be determined by X-ray powder diffraction. In particular, pharmaceutical compositions are described, where the X-ray powder diffraction pattern shows one or more discrete peaks for the active pharmaceutical ingredient. It is appreciated herein that the presence of one or more discrete peaks in the X-ray powder diffraction pattern is indicative of crystallinity. It is understood that X-ray powder diffraction may be performed as described herein, or using any conventional method and apparatus.

In another embodiment, pharmaceutical compositions are described, such as those of any of the preceding embodiments, wherein the active pharmaceutical ingredient is in at least a partially crystalline form, where the presence and degree of crystallinity may be determined by thermal analysis or calorimetry, such as using by differential scanning calorimetry (DSC), or differential thermal analysis (DTA). In particular, pharmaceutical compositions are described, where DSC or DTA curves show one or more discrete peaks or transition patterns for the active pharmaceutical ingredient. It is appreciated herein that the presence of one or more discrete peaks or transition patterns in the DSC or DTA curves is indicative of crystallinity. It is understood that DSC or DTA, or an equivalent technique, may be performed as described herein, or using any conventional method and apparatus.

In another embodiment, pharmaceutical compositions are described, such as those of any of the preceding embodiments, wherein at least one of the solid additives is in at least a partially crystalline form, where the presence and degree of crystallinity may be determined by X-ray powder diffraction. In particular, pharmaceutical compositions are described, where the X-ray powder diffraction pattern shows one or more discrete peaks for at least one of the solid additives. It is appreciated herein that the presence of one or more discrete peaks in the X-ray powder diffraction pattern is indicative of crystallinity. It is understood that X-ray powder diffraction may be performed as described herein, or using any conventional method and apparatus.

In another embodiment, pharmaceutical compositions are described, such as those of any of the preceding embodiments, wherein at least one of the solid additives is in at least a partially crystalline form, where the presence and degree of crystallinity may be determined by thermography or calorimetry, such as using by differential scanning calorimetry (DSC), or differential thermal analysis (DTA). In particular, pharmaceutical compositions are described, where DSC or DTA curves show one or more discrete peaks or transition patterns for at least one of the solid additives. It is appreciated herein that the presence of one or more discrete peaks or transition patterns in the DSC or DTA curves is indicative of crystallinity. It is understood that DSC or DTA, or an equivalent technique, may be performed as described herein, or using any conventional method and apparatus.

In another embodiment, pharmaceutical compositions are described, such as those of any of the preceding embodiments, wherein the majority of at least one of the active pharmaceutical ingredients is present as crystals in the solid suspension. In another embodiment, pharmaceutical compositions are described, such as those of any of the preceding embodiments, wherein the majority of at least one of the solid additives is present as crystals in the solid suspension.

In another embodiment, pharmaceutical compositions are described, such as those of any of the preceding embodiments, wherein the solid suspension is less than about 50% amorphous. In one variation, the solid suspension is less than about 20% amorphous. In another variation, the solid suspension is less than about 10% amorphous. In another variation, the solid suspension is less than about 5% amorphous. In another variation, the solid suspension is less than about 1% amorphous. As used herein, the term amorphous refers to solid forms that have little or no crystalline morphology or other molecular organization.

In another embodiment, pharmaceutical compositions are described, such as those of any of the preceding embodiments, wherein the solid suspension is greater than about 50% crystalline. In one variation, the solid suspension is greater than about 80% crystalline. In another variation, the solid suspension is greater than about 90% crystalline. In another variation, the solid suspension is greater than about 95% crystalline. In another variation, the solid suspension is greater than about 99% crystalline. It is appreciated that in each of the foregoing, there may be one or more crystalline morphologies of each component of the pharmaceutical compositions.

In another embodiment, pharmaceutical compositions are described, such as those of any of the preceding embodiments, wherein the solid suspension exhibits a crystallinity within 24 hours of preparation. In one variation, the solid suspension exhibits a crystallinity within 12 hours of preparation. In another variation, the solid suspension exhibits a crystallinity within 6 hours of preparation. In another variation, the solid suspension exhibits a crystallinity within 1 hour of preparation.

In another embodiment, pharmaceutical compositions are described, such as those of any of the preceding embodiments, wherein the active pharmaceutical ingredient has a solubility no greater than about 1 g/mL in a pharmaceutically acceptable organic solvent system is described. In one variation, the active pharmaceutical ingredient has a solubility no greater than about 100 mg/mL in a pharmaceutically acceptable organic solvent system. In another variation, the active pharmaceutical ingredient has a solubility no greater than about 10 mg/mL in a pharmaceutically acceptable organic solvent system.

In another embodiment, pharmaceutical compositions comprising an active pharmaceutical ingredient are described, such as those of any of the preceding embodiments, wherein the active pharmaceutical ingredient when unformulated has a solubility no greater than about 10 mg/mL in a pharmaceutically acceptable aqueous solvent system. In one variation, the active pharmaceutical ingredient when unformulated has a solubility no greater than about 1 mg/mL in a pharmaceutically acceptable aqueous solvent system. In another variation, the active pharmaceutical ingredient when unformulated has a solubility no greater than about 0.1 mg/mL in a pharmaceutically acceptable aqueous solvent system. In another variation, the active pharmaceutical ingredient when unformulated has a solubility no greater than about 1 μg/mL in a pharmaceutically acceptable aqueous solvent system.

In another embodiment, pharmaceutical compositions are described, such as those of any of the preceding embodiments, wherein the one or more active pharmaceutical ingredients account for between about 10% and about 50% by weight of the solid suspension. In one variation, the one or more active pharmaceutical ingredients account for between about 10% and about 40% by weight of the solid suspension. In another variation, the one or more active pharmaceutical ingredients account for between about 15% and about 35% by weight of the solid suspension.

It is to be understood that in each of the foregoing illustrative embodiments a single active pharmaceutical ingredient may be included, or that two active pharmaceutical ingredients may be included, or that a plurality of active pharmaceutical ingredients may be included in the formulations described herein. It is further to be understood that in each of the foregoing illustrative embodiments a single solid additive may be included, or that two solid additives may be included, or that a plurality of solid additives may be included in the formulations described herein.

As described herein, it has been unexpectedly found that the formulations described herein exhibit rapid disintegration, rapid dissolution, and/or rapid release rates, when compared to the corresponding unformulated active pharmaceutical ingredients. In one embodiment, the disintegration, rapid dissolution, and/or release rate of the active pharmaceutical ingredient from the formulations described herein is at least twice as rapid, at least three times more rapid, at least 5 times more rapid, or at least 10 times more rapid, compared to the corresponding unformulated active pharmaceutical ingredient when evaluated under similar or identical conditions. In another embodiment, pharmaceutical compositions are described, such as those of any of the preceding embodiments, wherein the solid suspension has a dissolution half-life in distilled water of about 6 hours or less. In one variation, the solid suspension has a dissolution half-life in distilled water of about 2 hours or less, or of about 1.5 hours or less.

In another illustrative embodiment, pharmaceutical compositions are described, such as those of any of the preceding embodiments, wherein the morphology of the solid suspension is characterized by an intimate mixture of active pharmaceutical ingredients and solid additives. In one aspect, the crystal size of each component in the solid suspension is small such that the bulk material exhibits a highly grained microstructure. In such a microstructure, when crystals of the same chemical composition are adjacent, they form separate grains or regions in the solid suspension, rather than combining to form a single larger crystal. Without being bound by theory, it is believed herein that such a microstructure positively contributes to the rapid dispersion and/or dissolution of the formulations described herein.

It is appreciated that the solid additives desirably have low toxicological potential, and have already been approved as a pharmaceutical or food ingredient. It is also understood that the solid additives desirably have hydrophilic properties. Without being bound by theory, it is believed herein that the combination of those hydrophilic properties, the intimate mixture of the active pharmaceutical ingredients and the solid additives, and the crystalline nature of each component each leads to the enhancement of the dissolution rate of the active pharmaceutical ingredient. In addition, and without being bound by theory, it is believed herein that the combination of the intimate mixture of the active pharmaceutical ingredients and the solid additives, and the crystalline nature of each component also leads to the enhancement of stability of the formulation.

Also described herein are processes for preparing the solid suspensions described herein. In one embodiment, the solid suspensions are prepared by extrusion. In one aspect, the process includes the steps of mixing about 5-95% by weight of the active pharmaceutical ingredient with about 95-5% by weight of the one or more pharmaceutically acceptable water soluble solid additives; heating said mixture comprising the active pharmaceutical ingredient and the one or more solid additives to a temperature that is about at or above the melting point of at least one of the solid additives; and extruding the heated mixture to form the solid suspension. In one variation, the about 5-95% by weight of the active pharmaceutical ingredient is added separately from the about 95-5% by weight of the one or more pharmaceutically acceptable water soluble solid additives. It is appreciated that the active pharmaceutical ingredient may be added first and heated prior to the addition of the one or more water soluble solid additives, or in the alternative the one or more water soluble solid additives may be added first and heated prior to the addition of the active pharmaceutical ingredient.

Illustrative extrusion apparatus are described herein, though it is to be understood that any conventional extrusion apparatus may be used to prepare the formulations described herein. In one aspect, the extrusion process is performed with high torque, such that the extrusion apparatus transfers sufficient energy to the mixture of active pharmaceutical ingredients and solid additives. In one variation, the extrusion process is performed with high shear, such that the extrusion apparatus transfers sufficient energy to the mixture of active pharmaceutical ingredients and solid additives. Without being bound by theory, it is believed herein that high torque, and/or high shear used in the processes described herein, each may contribute to potentially high active pharmaceutical ingredient loads of the solid suspensions described herein. In addition, and without being bound by theory, it is believed herein that high torque, and/or high shear used in the processes described herein, each may contribute to potentially rapid dissolution rates of the solid suspensions described herein. In addition, and without being bound by theory, it is believed herein that high torque, and/or high shear used in the processes described herein, each may contribute to the crystallinity exhibited by the solid suspensions described herein. Such crystallinity includes both the propensity and rate that the crystallinity develops, as described herein, and well as the overall nature of the microcrystalline structure, grain size, and grain arrangement of the components forming the solid suspensions described herein.

In another aspect, the extrusion process is performed at a temperature that is at or above the melting temperature of at least one of the solid additives. In one variation, the extrusion process is performed at a temperature that is at or above the melting point of the combination of all of the solid additives. In another variation, the extrusion process is performed at a temperature that is at or above the melting point of the highest melting solid additive. In another variation, the extrusion process is performed at a temperature that is below the melting temperature of at least one of the active pharmaceutical ingredients. In another variation, the extrusion process is performed at a temperature that is below the melting temperature of the combination of the active pharmaceutical ingredients. In another variation, the extrusion process is performed at a temperature that is below the lowest melting temperature of any of the active pharmaceutical ingredients.

The solid suspensions described herein may be processed in any conventional manner to prepare solid dosage forms, including but not limited to tablets, capsules, dispersible powders, and the like. It is to be understood that additional carriers, diluents, and/or excipients may be added to the solid suspensions described herein to prepare the dosage form. Illustrative conventional processing is described in for example U.S. Pat. Nos. 4,310,543, 4,525,339, 4,892,742, 5,190,748, 5,318,781, 5,393,765, 6,008,228, 6,350,786, 6,492,530, and 7,014,866, the disclosures of which are incorporated herein by reference.

### EXAMPLES

### Materials

The following materials were used as received from commercial suppliers: griseofulvin (Hawkins, Minneapolis, Minn., USA), mannitol (Pearlito 150 C, Roquette, Lestrem, France), adonitol (Alfred Aesar, Karlsruhe, Germany), fructose (Aldrich, Milwaukee, Wis., USA), glucose (Merck, Rahway, N.J., USA), sorbitol (ICI Americans, Willington, Del., USA) and xylitol (Spectrum, Gardena, Calif., USA), phenytoin (Spectrum, Gardena, Calif., USA) and spironolactone (Hawkins, Minneapolis, Minn., USA). All substances were US Pharmacopeia (USP) grade. The active pharmaceutical ingredients used in this study are known in the pharmaceutical field to have low solubility and slow dissolution rates. As model compounds, they represent a viable test for the solid suspension methodology presented.

### Example Methods

### Extrusion

The dry powder materials were premixed in a beaker and subsequently transferred to the ram feeder of the extruder (Haake MiniLab, Thermo Electron, Newington, N.H., USA). Approximately 7 g powder material was divided into four different feeding steps which were carried out one after another. The materials were mixed in the extruder and subsequently extruded through a 1 mm diameter die. The extrudates were cooled on aluminum foil to 25° C. and then stored for further characterization at 25° C., 60% relative humidity (RH) for 24 h as well as at 40° C., 75% RH for 28 d and 90 d. These are typical stress-storage conditions that may be used for stability testing.

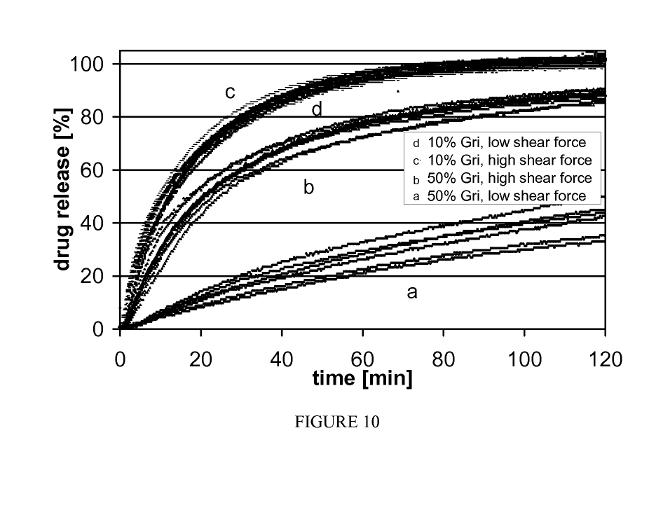

Pre-mixed, dry powder materials (10% griseofulvin in α-mannitiol or 50% griseofulvin in α-mannitiol) were extruded using a production scale extruder (Leistritz Mikro GL 27-28D, Leistritz, Nuermber, Germany). The extrusion process was carried out at the melting point of the α-mannitiol using a powder feed rate of 40 g/min and a screw speed 100 rpm. The shear rate was varied on two levels during extrusion by varying the barrel length, the number of die holes and screw configuration. The extrudates were characterized by a dissolution test in accordance to the preliminary experiments (see FIG. 10).

### Dissolution

The dissolution tests were performed in a paddle apparatus (VK7030, Varian, Cary, N.C., USA) in accordance with the USP at 50 rpm. Six samples of each batch were tested in water at 37° C. as dissolution media. For the dissolution test, the extrudates were cut in small pieces of approximately 2 mg. The active pharmaceutical ingredient release was quantified with a UV-photometer (DU 640, Beckman, Fellerton, USA; Cary 300, Varian, Victoria, Australia) using different wavelengths (griseofulvin 296 nm, phenytoin 220 nm and spironolactone 243 nm) for 120 min using a cuvette with a 50 mm path length.

### Differential Scanning Calorimetry

Thermograms were obtained using a differential scanning calorimeter (Q10, TA Instruments, New Castle, Del., USA). Accurately weighed samples of approximately 2 mg were hermetically sealed in aluminum pans and heated from −25 to 250° C. at 10 K/min. Dry nitrogen with a flow rate of 50 ml/min was used to purge the sample compartment of the oven. Each sample was measured in duplicate.

### X-Ray Diffraction

The crystal structure was characterized by X-Ray diffraction (LabX XRD6000, Shimadzu, Columbia, Md., USA). A Cu Ka radiation point source (k=1.5406 A) was operated at 40 kV and 30 mA. The powdered samples were placed in aluminum holders and measured in the reflection mode from 10 to 40° 2θ. The scanning rate was 5°/min using a sampling pitch of 0.02°. Each sample was measured in duplicate.

### Example Formulations and Process Examples

The three active pharmaceutical ingredients, griseofulvin (Gri), phenytoin (Phe) and spironolactone (Spi), were chosen based on their low solubilities and their high UV absorptions in aqueous solution. They were used as model active pharmaceutical ingredients apart from their therapeutic indication or concentration in the pharmaceutical dosage form. Mannitol is a known excipient in pharmaceutical products and was chosen for its low toxicity and high solubility.

This study is structured in two parts. The first part is a proof of the “solid suspension” concept using the three different model active pharmaceutical ingredients at 10% (w/w) load (tab. 1, Gri 10, Phe 10, Spi 10). In the second part one these active pharmaceutical ingredients was picked to investigate storage stability and the feasibility of manufacturing a solid suspension with a high (50% w/w) load (TABLE 1, Gri50).

TABLE 1
Powder formulations

| substance | Gri10 | Phe10 | Spi10 | Gri50 |
|---|---|---|---|---|
| griseofulvin | 10 | | | 50 |
| phenytoin | | 10 | | |
| spironolactone | | | 10 | |
| mannitol | 90 | 90 | 90 | 50 |
| lactic acid | 90 | | | |
| xyitol | 90 | | | |

### Extrusion

The active pharmaceutical ingredient and the excipient were co-processed using a laboratory scale co-rotating twin screw extruder (Haake MiniLab). The extrusion barrel of the extruder has only one heating zone in comparison to most production scale screw extruders which have several. Therefore, the extrusion die was locked, and the feeding, mixing and extrusion steps were completed in separate steps rather than simultaneously (TABLE 2).

TABLE 2
Process parameters extrusion process

| | time | temperature | screw speed |
|---|---|---|---|
| step | [min] | [° C.] | [rpm] |
| feeding | 3 | 165 | 360 |
| mixing | 15 | 158 | 200 |
| extrusion | 1 | 165 | 200 |

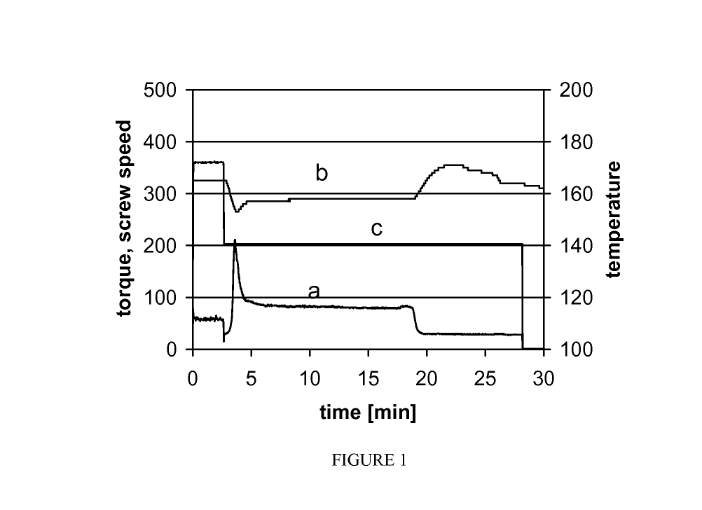

The feeding process was performed at the melting temperature of mannitol (165° C.) in order to plasticate the powder material. During feeding, the screw speed was set to 360 rpm in order to accelerate the feeding of the powder. The feeding procedure was completed in 3 min (FIG. 1). During the mixing phase, the screw speed was decreased to 200 rpm which was found adequate in several pretests. The barrel temperature was also decreased in order to increase the frictional forces on the extrudate by increasing the viscosity. Therefore, torque of the extrusion screws increased after an equilibration period of an additional 1 min. The material was then mixed for 15 min in order to produce a homogeneous mixture. Subsequently, the barrel temperature was increased to 165° C. with an equilibration time of 7 min to eliminate any potential clogging of the die.

### Active Pharmaceutical Ingredient Release

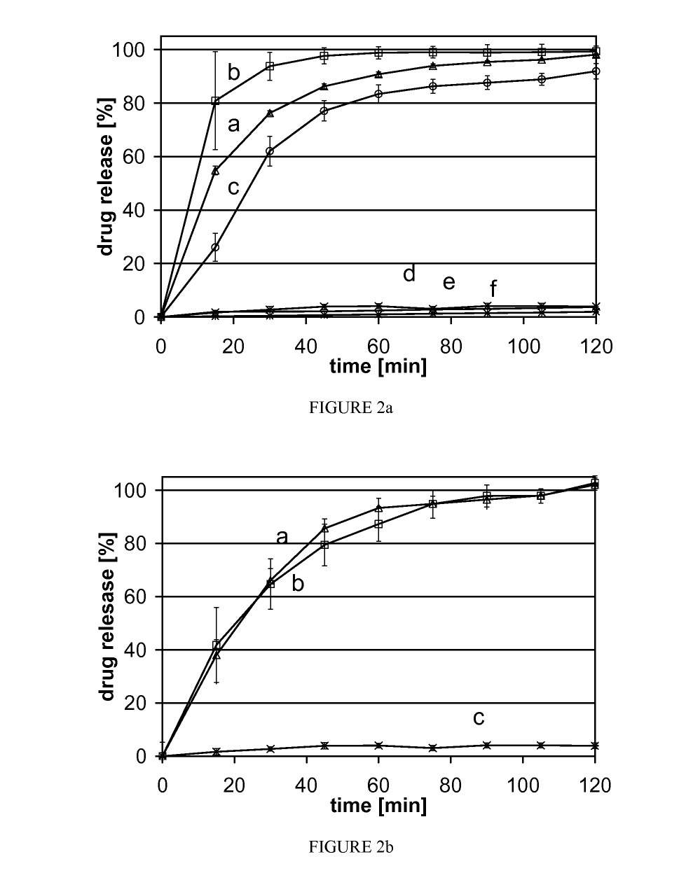

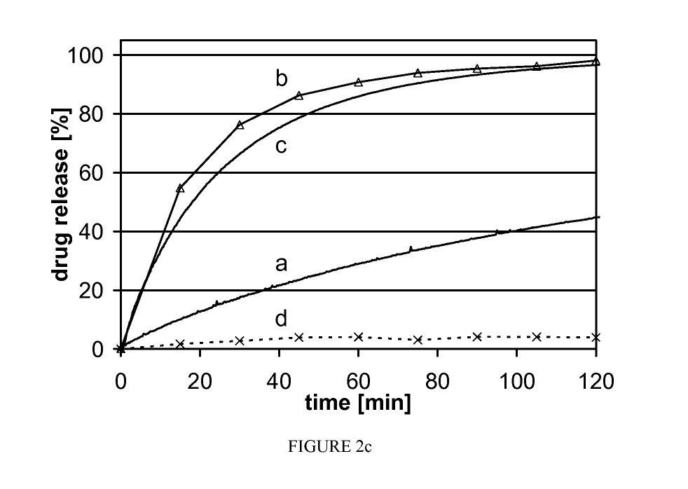

The active pharmaceutical ingredient release from the extrudates of all three active pharmaceutical ingredients was almost complete in two hours (FIG. 2a). Comparatively, it took six days for the pure griseofulvin to attain 50% release (data in the FIGURE is cut at 120 min). The data indicate that the increase in the dissolution rate obtained by the solid suspension described herein is on the order of 500-fold (based on the t_{1/2}). It has been reported that such a dramatic magnitude of enhancement in the dissolution rate is only achieved with the traditional solid dispersion approach requiring the less desirable formation of an amorphous sample.

FIG. 2b shows the dissolution profiles from extrudates with high active pharmaceutical ingredient loads of 50% griseofulvin and the profile for pure active pharmaceutical ingredient. The active pharmaceutical ingredient release from this extrudate is marginally slower than that from the extrudates containing 10% active pharmaceutical ingredient load. These observations support the generality of the methods described herein and indicate that such a preparation of a solid suspension is not limited by the active pharmaceutical ingredient load. In other words, the ability to produce the desired dissolution rate enhancement at high and low active pharmaceutical ingredient loads implies that the methodology will be applicable to a wide variety of active pharmaceutical ingredients, including those of high potency (low load) as well as those requiring higher doses (high load). It is appreciated that from a manufacturing perspective, the same procedure can be applied to obtain different doses of the same active.

The extrudate containing 10% griseofulvin and 90% xylitol has a fast dissolution rate which is similar to that of the formulation with 10% griseofulvin and 90% mannitol. The active pharmaceutical ingredient release from the formulation containing L-(+)-lactic acid is slower than the mannitol and xylitol formulations. However, it is still much faster than the active pharmaceutical ingredient release from the pure active pharmaceutical ingredient. The dissolution rate of the extrudate can be modified by the choice of excipient (FIG. 2c).

The fresh and the stored extrudates have statistically the same active pharmaceutical ingredient release rates (a=0.05) which indicates a stable formulation.

### Crystallinity

The results presented above demonstrate that the solid suspension approach introduced here produces the desirable enhancement in dissolution rate of similar magnitude as that obtained from traditional (amorphous, thermodynamically unstable) solid dispersions. However, it is appreciated that a major advantage of the solid suspension compared to the solid dispersion may be based on the crystalline structure of the extrudate which makes the dosage form more thermodynamically stable. Therefore, crystallinity of the extrudate was determined by differential scanning calorimetry as well as X-Ray diffraction.

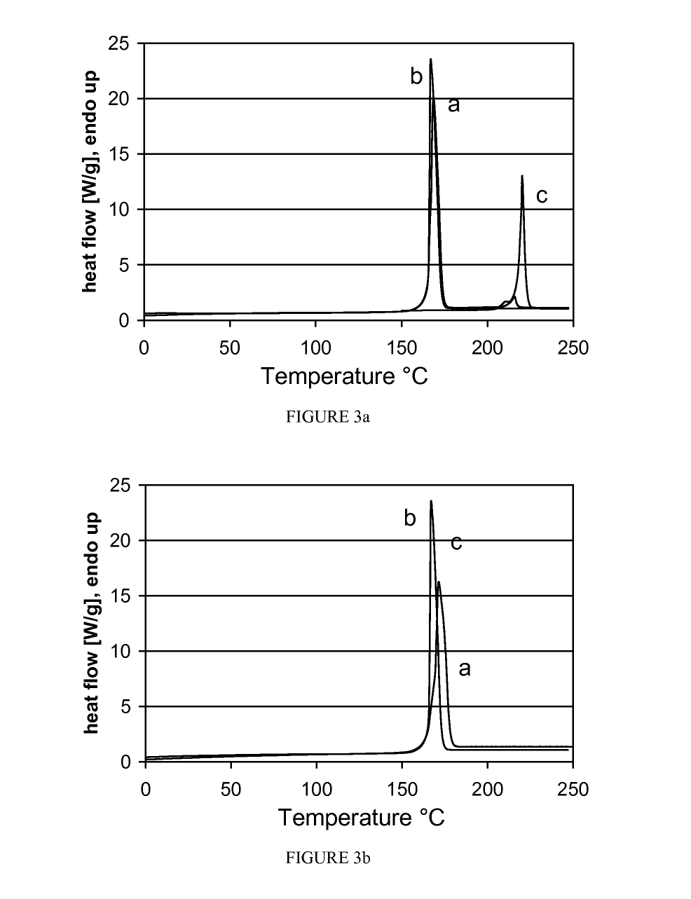

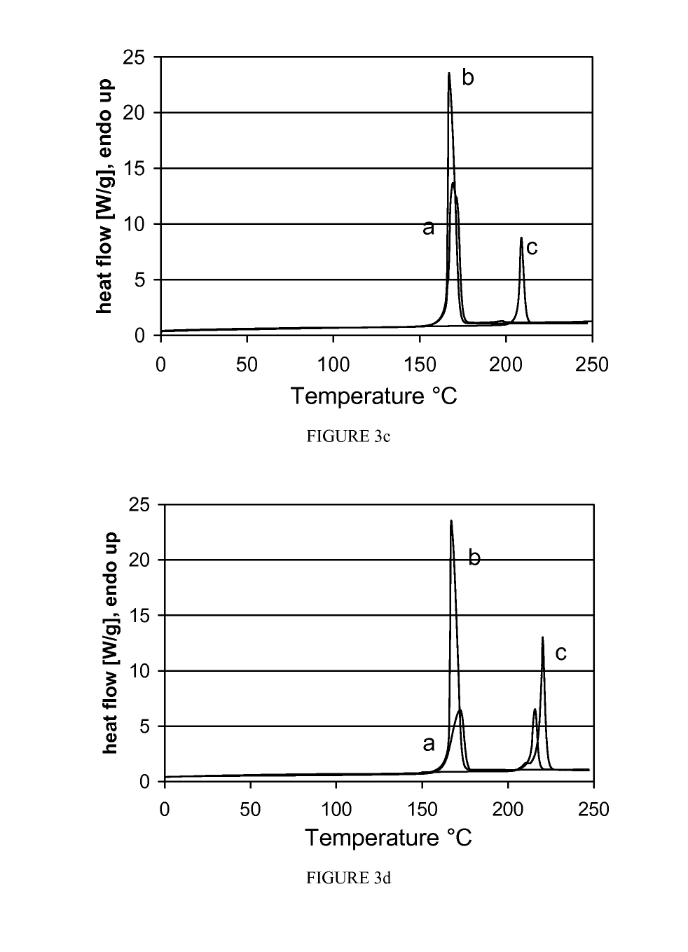

The melting temperature of mannitol in the extrudate is the same as the melting temperature of pure α-mannitol. The mannitol melting peak for the extrudate is broader which can be attributed to the presence of active pharmaceutical ingredient. The melting point depression for the active pharmaceutical ingredients in the extrudates compared to the pure active pharmaceutical ingredients was caused by the presence of mannitol which acted as an impurity in the molten (liquid) phase (FIGS. 3a, 3b, 3c, and 3d). Based on the obtained thermograms, amorphous solid dispersions, co-crystals and eutectic mixtures can be excluded as reasons for the rapid active pharmaceutical ingredient release. The melting point of phenytoin could not be determined because it is very close to the boiling point of the mannitol (FIG. 3b).

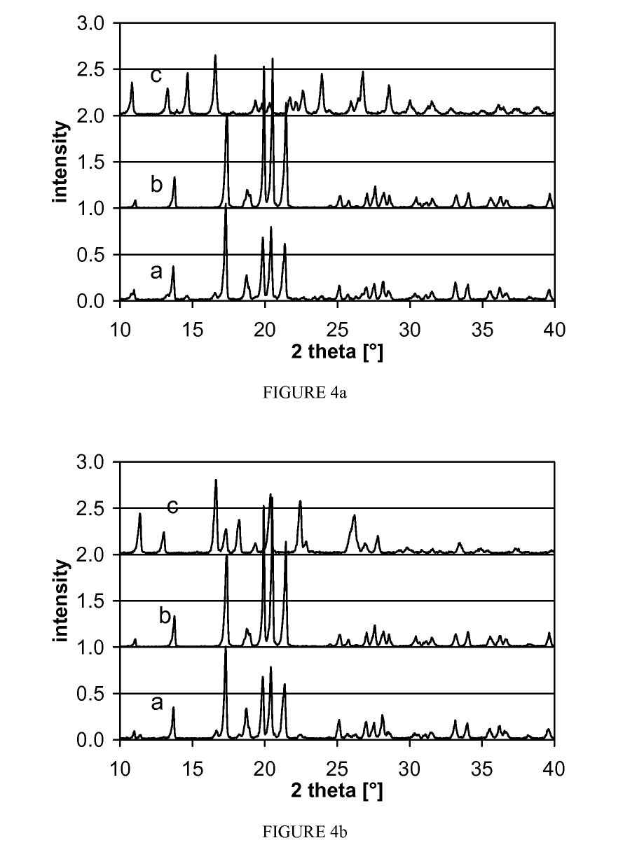

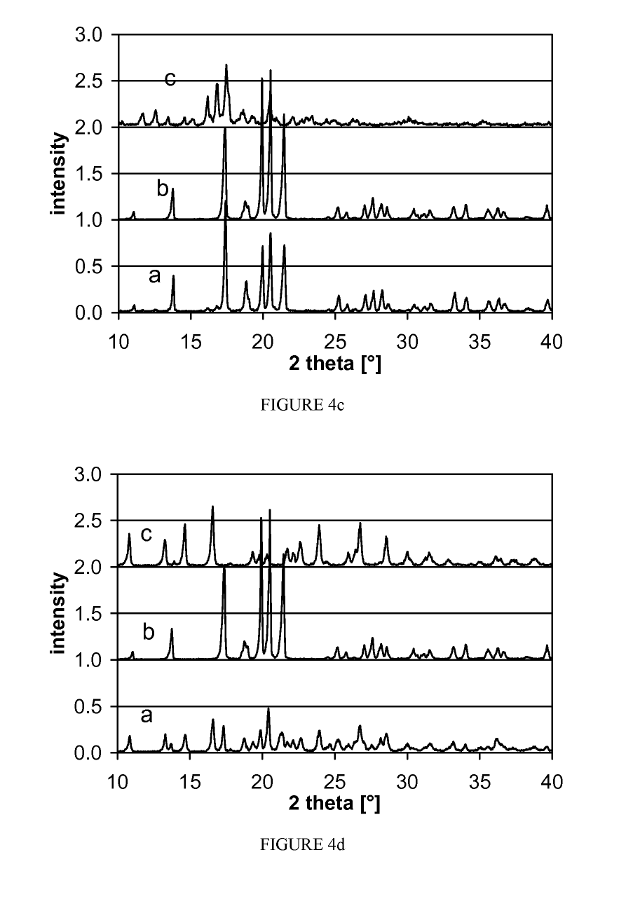

All peaks in the diffraction pattern of the extrudates were explainable by the diffraction pattern of active pharmaceutical ingredient or by the diffraction pattern of a-mannitol (FIGS. 4a, 4b, 4c and 4d). This demonstrates that the extrudate is a physical mixture of crystalline active pharmaceutical ingredient and a-mannitol.

In additional embodiments of the invention, solid suspension extrudates were prepared from griseofulvin and sorbitol, griseofulvin and fructose, and griseofulvin and sucrose.

### Solid Additive Examples

### Carbohydrates

Additional sugars were investigated in the present study. Glucose and fructose are two sugars, which appear to also possess the advantageous properties described above. Glucose and fructose are monosaccharides contained in several oligo- and polysaccharides, making them suitable illustrative examples for this investigation.

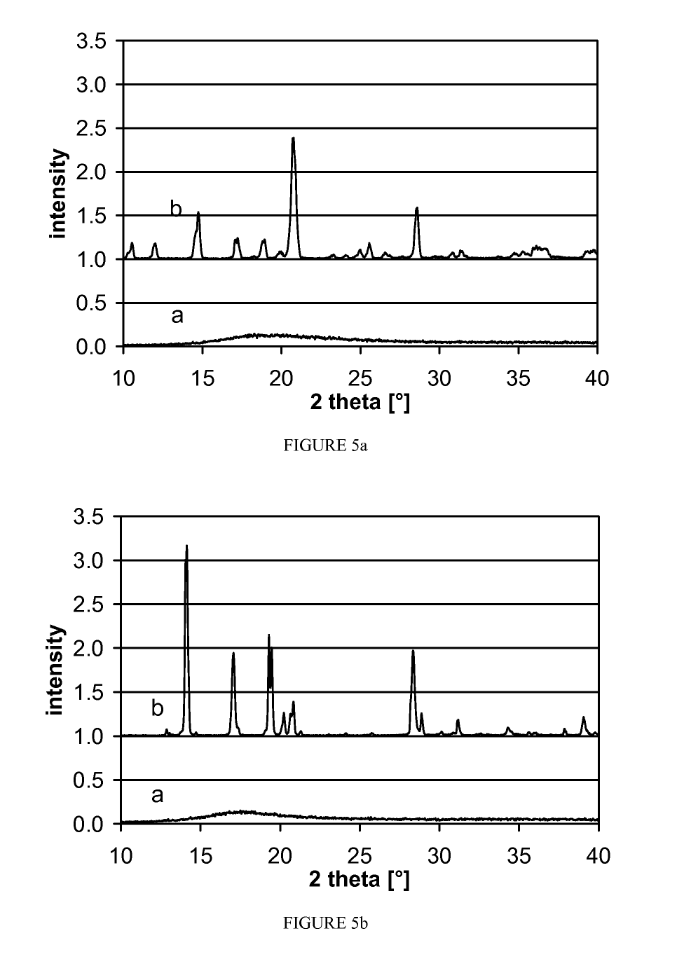

The X-Ray diffraction (FIGS. 5a, 5b) patterns indicate that neither glucose nor fructose crystallized after extrusion. Both substances remained as amorphous solids for more than 24 h. The reason for this may be the cyclical molecular structure which prevents rapid orientation of the molecule during crystallization. Accordingly, solid suspensions of glucose and fructose were not prepared.

### Polyhydroxy Compounds

In another illustrative embodiment, a group of the polyols with linear molecular structure are described. Another member of the polyols is sorbitol, a stereoisomer of mannitol, which is found to be a suitable excipient.

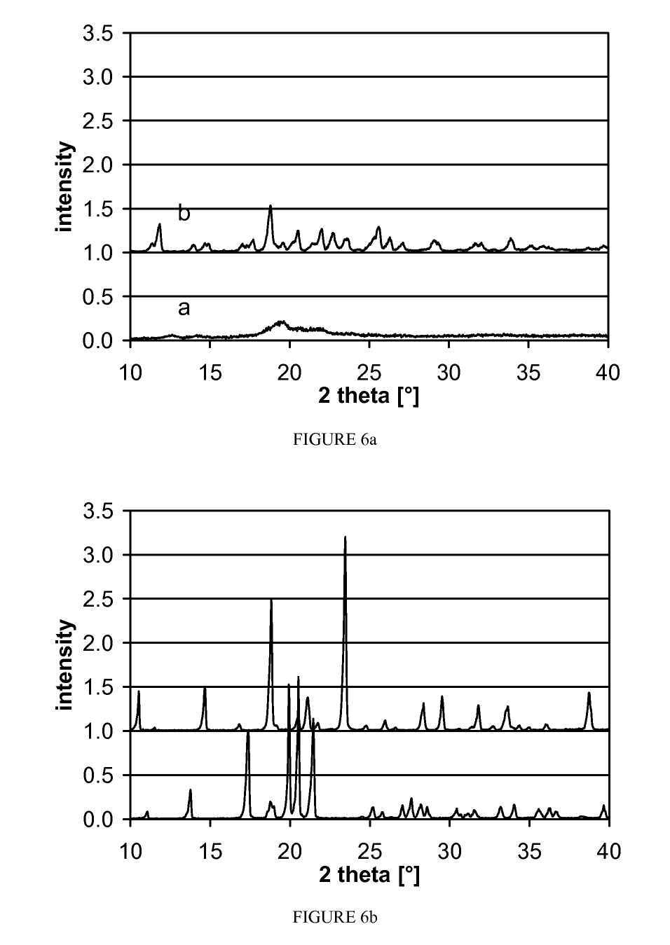

Sorbitol does not crystallize as fast as mannitol and was still predominantly amorphous after 24 h (FIGS. 6a, 6b). The different crystallization kinetics of the isomers suggests that the crystallization kinetic is related to the stereochemical structure. In contrast to sorbitol, mannitol has a symmetric molecular structure, which increases the probability of the correct orientation of each molecule during crystallization. Without being bound by theory, this may be the reason for the faster crystallization of the mannitol as compared to Sorbitol.

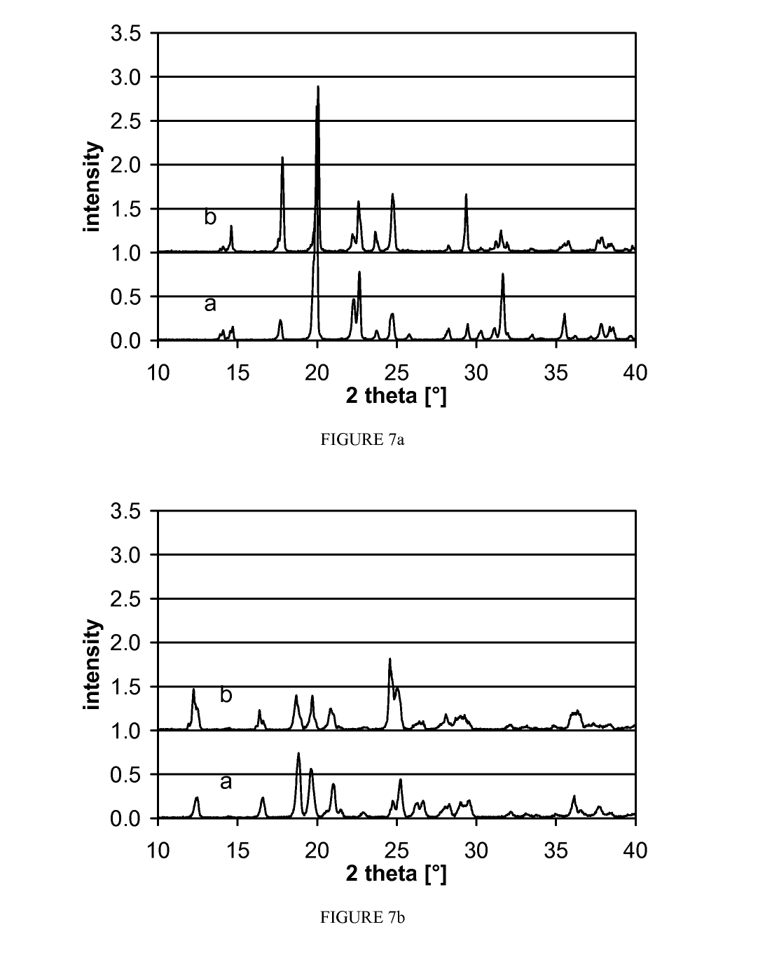

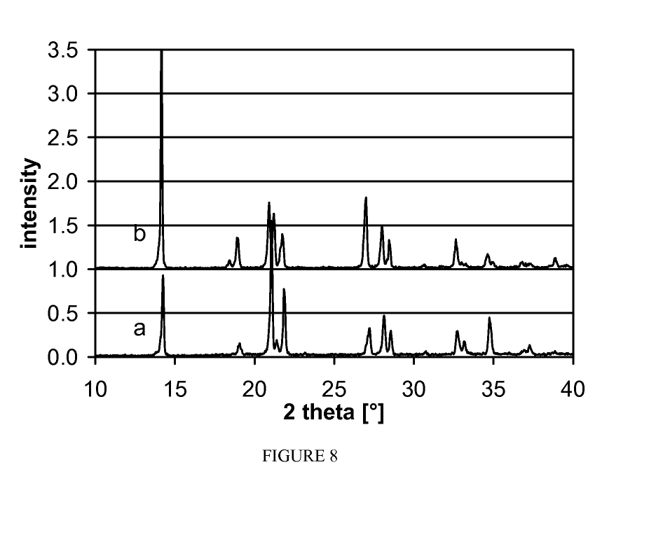

In another illustrative embodiment, two other polyols are described, the symmetric xylitol and the asymmetric adonitol. The correlation of the crystallization kinetics with the symmetric or asymmetric molecular structure was not established for these substances (FIGS. 7a, 7b). However, xylitol and adonitol have a lower molecular weight than mannitol and sorbitol. Without being bound by theory, it is appreciated that smaller molecules may have in general higher molecular mobility and a tendency to crystallize faster than large molecules with a similar chemical structure. This may be the reason for the rapid crystallization of the asymmetric adonitol.

### Hydroxy Carboxylic Acids

If the molecular size affects the crystallization kinetic, small molecules should crystallize quickly regardless of their chemical structure. In one variation, L-(+)-Lactic acid is described as a hydrophilic substance with a low molecular weight.

The crystallization of L-(+)-lactic acid was very rapid and was completed within 24 h supporting the hypothesis (FIG. 8).

In another embodiment, xylitol and lactic acid are described in the preparation of extrudates with a load of 10% griseofulvin. The extrusion temperature was set to 100° C. for xylitol and 53° C. for lactic acid. These temperatures are much lower than the temperature used with mannitol in the previous study. Without being bound by theory, it is appreciated that lower temperatures may reduce thermal stress on the active pharmaceutical ingredient in the formulation. Therefore, xylitol and lactic acid may be better suited than mannitol, in terms of thermal stability of the active pharmaceutical ingredient during processing, for formation of solid solutions of active pharmaceutical ingredients with greater sensitivity to temperature during formulation.

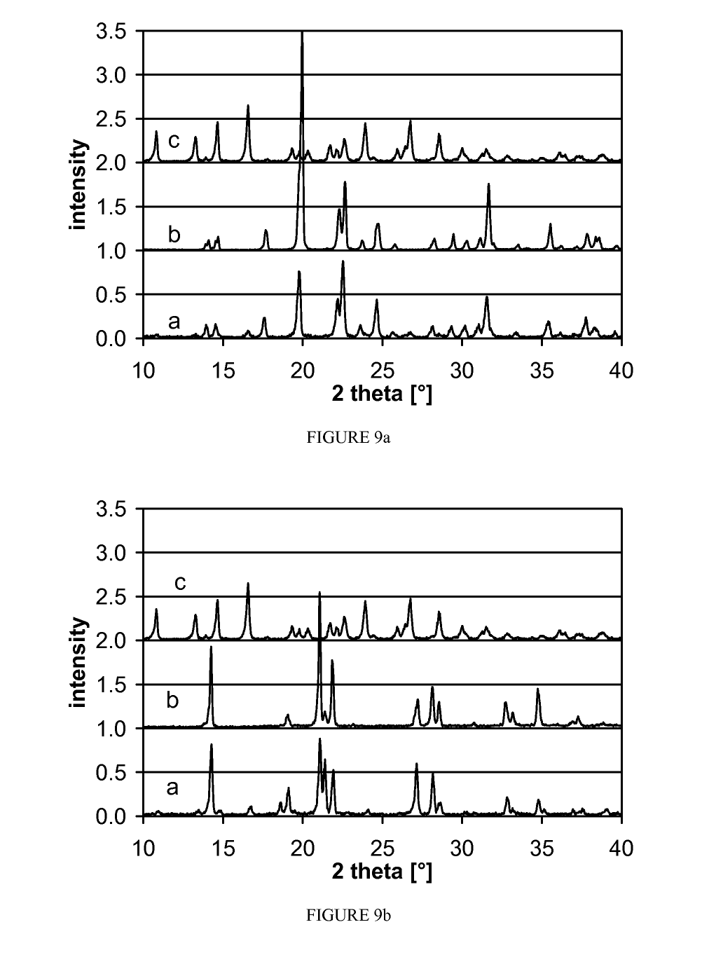

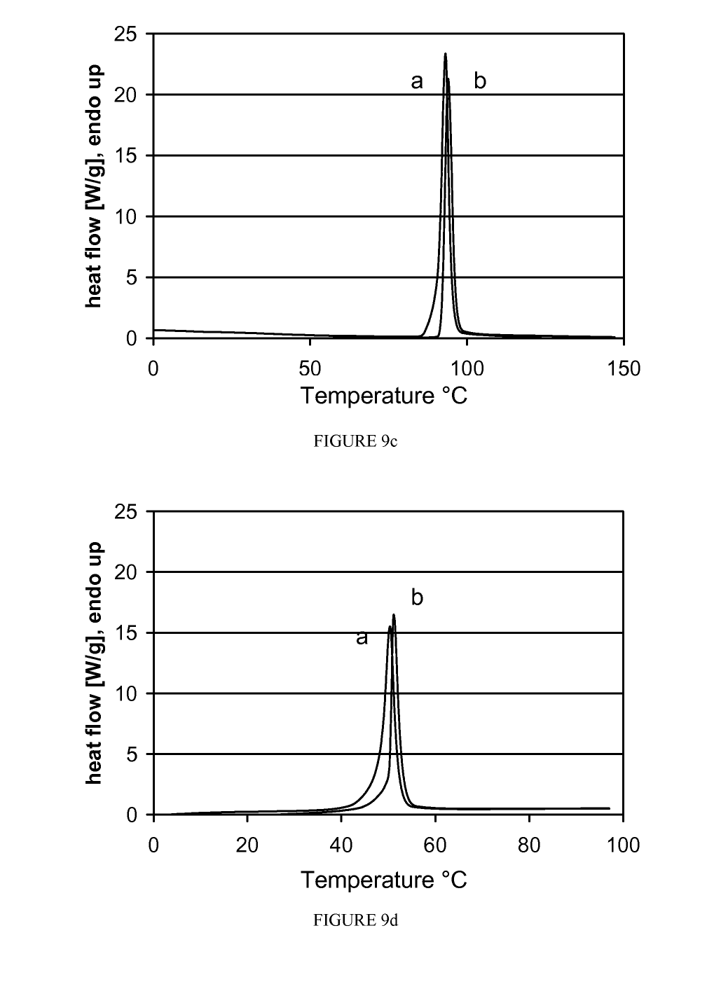

The peaks in the X-Ray diffraction pattern of the extrudates (FIGS. 9a, 9b) can be satisfactorily attributed to either the excipient (xylitol, lactic acid) or the active pharmaceutical ingredient (griseofulvin). This indicates that the extrudate is a crystalline mixture of the two substances, which is one of the desired attributes of the formulation described herein. The melting point of the excipients in the extrudate is marginally depressed in comparison to the pure excipient (FIGS. 9c, 9d). Without being bound by theory, this depression may be attributed to the presence of the active pharmaceutical ingredient which acts as a low level impurity in the excipient. The melting point of griseofulvin was not investigated in the extrudate because it is above the boiling point of xylitol and lactic acid. The hermetically sealed pans might be destroyed below the melting point of the griseofulvin by the vapor pressure of the xylitol or the mannitol. The thermograms show the absence of a eutectic and the non amorphous, i.e. crystalline, properties of the formulation.

The preparation of the crystalline mixtures by hot melt extrusion is described as an effective way of increasing the dissolution rate of poorly soluble active pharmaceutical ingredients. Though counter intuitive, the magnitude of enhancement of the dissolution rate is comparable to known amorphous solid dispersions. In certain embodiments xylitol, L-(+)-lactic acid, mannitol are suitable for use in the manufacturing of intimate crystal mixtures by hot melt extrusion. It has also been observed herein, that the crystallization kinetic, which, without being bound by theory, may be related to the molecular size and stereochemistry of the molecule, may be a useful factor for choosing a suitable excipient for preparing the solid suspensions described herein. Also described herein are methods for preparing thermodynamically stable dosage forms with a high active pharmaceutical ingredient load.

1.-60. (canceled)

61. A pharmaceutical composition comprising a solid suspension including about 5-95% by weight of an active pharmaceutical ingredient, and about 95-5% by weight of one or more pharmaceutically acceptable water soluble solid additives; wherein at least one of the solid additives has a melting temperature less than the melting temperature of the active pharmaceutical agent; at least a portion of the active pharmaceutical ingredient is present as crystals in the solid suspension; and at least a portion of the solid additives is present as crystals in the solid suspension.

62. The pharmaceutical composition of claim 61 wherein the one or more solid additives are selected from the group consisting of polyhydroxy compounds, hydroxy carboxylic acids, polyhydroxy carboxylic acids, and combinations thereof.

63. The pharmaceutical composition of claim 61 wherein the one or more solid additives are selected from the group consisting of reduced carbohydrates, sugar alcohols, and hydroxy carboxylic acids, and combinations thereof.

64. The pharmaceutical composition of claim 61 wherein at least one of the solid additives is selected from the group consisting of arabitol, erythritol, xylitol, sorbitol, mannitol, lactic acid, malic acid, tartaric acid, citric acid, adonitol, and lactitol.

65. The pharmaceutical composition of claim 61 wherein at least one of the solid additives is selected from the group consisting of xylitol, mannitol, and lactic acid.

66. The pharmaceutical composition of claim 61 wherein the active pharmaceutical ingredient has a melting point of at least about 100° C.

67. The pharmaceutical composition of claim 61 wherein the active pharmaceutical ingredient has a melting point of at least about 200° C.

68. The pharmaceutical composition of claim 61 wherein the active pharmaceutical ingredient has a solubility no greater than about 1 g/mL in a pharmaceutically acceptable organic solvent system.

69. The pharmaceutical composition of claim 61 wherein the active pharmaceutical ingredient has a solubility no greater than about 10 mg/mL in a pharmaceutically acceptable organic solvent system.

70. The pharmaceutical composition of claim 61 wherein the active pharmaceutical ingredient has a solubility no greater than about 10 mg/mL in a pharmaceutically acceptable aqueous solvent system.

71. The pharmaceutical composition of claim 61 wherein the active pharmaceutical ingredient has a solubility no greater than about 0.1 mg/mL in a pharmaceutically acceptable aqueous solvent system.

72. The pharmaceutical composition of claim 61 wherein the solid suspension further comprises a second active pharmaceutical ingredient.

73. The pharmaceutical composition of claim 61 wherein the active pharmaceutical ingredient is selected from the group consisting of ibuprofen, paclitaxol, griseofulvin, itraconazole, phenytoin, spironolactone, and combinations thereof.

74. The pharmaceutical composition of claim 61 wherein the solid suspension comprises at least two water soluble additives.

75. The pharmaceutical composition of claim 61 wherein the solid suspension has a dissolution half-life in water of about 6 hours or less.

76. The pharmaceutical composition of claim 61 wherein the majority of the active pharmaceutical ingredient is present as crystals in the solid suspension.

77. The pharmaceutical composition of claim 61 wherein the majority of at least one solid additive is present as crystals in the solid suspension.

78. The pharmaceutical composition of claim 61 wherein the solid suspension is less than about 50% amorphous.

79. The pharmaceutical composition of claim 61 wherein the solid suspension is less than about 5% amorphous.

80. A process for preparing the solid suspension of claim 61, the process comprising the steps of:
mixing the active pharmaceutical ingredient with the one or more pharmaceutically acceptable water soluble solid additives;
heating said mixture comprising the active pharmaceutical ingredient and the one or more solid additives to a temperature that is about at or above the melting temperature of at least one of the solid additives; and
extruding the heated mixture to form the solid suspension.

81. The process of claim 80 wherein the mixture is heated to a temperature that is about at or above the melting temperature of at least one of the solid additives and below the melting temperature of the active pharmaceutical agent.
